(19) United States
(12) Patent Application Publication
(10) Pub. No.: US 2023/0265509 A1
(43) Pub. Date: Aug. 24, 2023

(54) NOVEL TARGETS FOR THE TREATMENT AND DIAGNOSIS OF PATIENTS WITH ATHEROSCLEROSIS AND ENHANCED RISK OF END ORGAN DAMAGE

(71) Applicant: David R. Kaplan, Shaker Heights, OH (US)

(72) Inventor: David R. Kaplan, Shaker Heights, OH (US)

(21) Appl. No.: 17/676,072

(22) Filed: Feb. 18, 2022

(51) Int. Cl. C12Q 1/6883; G01N 33/68

(52) U.S. Cl. CPC C12Q 1/6883; G01N 33/6872; C12Q 2600/158; C12Q 2600/112; C12Q 1/6844

(57) ABSTRACT

The invention is directed to a method to test a subject having, or at risk for having, atherosclerosis, the method comprising obtaining peripheral blood cells, particularly mononuclear cells, from the subject and assaying the cells for gene expression of one or more components of the STING (stimulator of interferon genes) pathway relative to an appropriate baseline control. The invention is also directed to a composition comprising a peripheral blood cell that is co-stained for a marker that is specific for the peripheral blood cell and for a marker that is specific for gene expression of a component of the STING pathway, the peripheral blood cell being derived from a subject having or at risk for having atherosclerosis. Peripheral blood cells include B lymphocytes, T lymphocytes, monocytes and natural killer cells. Specific subsets of lymphocytes include CD4⁺ and CD8⁺ T lymphocytes.

### FIELD OF THE INVENTION

The invention is directed to a method to test a subject having, or at risk for having, atherosclerosis, the method comprising obtaining peripheral blood cells, particularly mononuclear cells, from the subject and assaying the cells for gene expression of one or more components of the STING (stimulator of interferon genes) pathway relative to an appropriate baseline control.

The invention is also directed to a method for testing a compound for its effect on atherosclerosis by administering a compound to a subject having, or at risk for having, atherosclerosis and assessing the effect of the compound on the development, progression, or severity of the disease, the compound being an inducer or inhibitor of gene expression of one or more components of the STING pathway.

The invention is also directed to a method for treating a subject having, or at risk for having, atherosclerosis comprising administering an inducer or inhibitor of gene expression of one or more components of the STING pathway to the subject.

The invention is also directed to a composition comprising a peripheral blood cell that is co-stained for a marker for the peripheral blood cell and for a marker for gene expression of a component of the STING pathway, the peripheral blood cell being derived from a subject having or at risk for having atherosclerosis.

The invention is also directed to kits that contain antibodies to cell lineage markers on peripheral blood cells and antibodies to one or more STING pathway components, to be used to test a subject for atherosclerosis or the effect of an agent on gene expression of one or more components of the STING pathway in peripheral blood cells in a sample to assess the effect of the agent on one or more components of the STING pathway in the peripheral blood cells.

Peripheral blood cells include B lymphocytes, T lymphocytes, monocytes and natural killer (NK) cells. Specific subsets of lymphocytes include CD4⁺ and CD8⁺ T lymphocytes. Components of the STING pathway include STING, phospho-STING, Akt, phospho-Akt, IRF3, phospho-IRF3, TBK1 (TANK-binding kinase 1), phospho-TBK1, ULK1, phospho-ULK1, cGAS, NLRP3 (NACHT, LRR, and PYD domains-containing protein 3), RelA, phospho-RelA, BDNF (brain derived neurotrophic factor), VEGF, MyD88, Trex1, AIM2, Caspase1, SURF4, STEEP1, STIM1, HER2, Stat6 and Src.

The atherosclerosis includes coronary artery disease, carotid artery disease, aortic disease, and disease of the peripheral arteries, such as, arteries of the upper and lower extremities and the renal arteries.

### BACKGROUND OF THE INVENTION

### Molecular Correlates of Atherosclerosis

Atherosclerosis is characterized by the deposition of low-density lipoprotein in the arterial intima of arteries which triggers recruitment of immune cells from the blood to the vessel walls and subsequent inflammatory cascades. The lipids are taken up by smooth muscle cells and monocytes/macrophages. The macrophages are induced to produce and secrete pro-inflammatory cytokines and chemokines. These processes progress to eventual unresolved inflammation, impaired efferocytosis, cell necrosis, micro-vessel formation, fibrous cap thinning, and destabilization of the lesions. The immune cells that are involved in this process include monocytes/macrophages, T and B lymphocytes, neutrophils, endothelial cells, and smooth muscle cells (1).

As atherosclerosis progresses, calcium is deposited in the arterial lesions. Most importantly, the degree of calcium deposition in the arterial walls is correlated with the risk of atherosclerosis especially as indicated by coronary artery calcium depositions that are associated with cardiovascular disease (2).

Type I interferon is a well-known inflammatory pathway mediator that has been implicated in the pathophysiology of atherosclerosis (1). The source of the cytokines has been found to be macrophages and plasmacytoid dendritic cells in the atherosclerotic lesions (3,4). Additionally, the role of type I interferon in atherosclerosis has been inferred by studies in murine models instead of by direct observations of samples from human tissue (1-4).

Some studies of human atherosclerosis have focused on the analysis of carotid plaque specimens obtained at the time of carotid endarterectomy Immunohistochemistry of these samples has shown the presence of activated T cells with expression of HLA-Dr and VLA-1 (5), the expression of inducible nitric oxide synthase and IL10 in macrophages and smooth muscle cells (6), and the presence of HSP70 in atheroma macrophages (7). An alternative source of tissue has been aortic lesions obtained at autopsy. In one study secretory phospholipase A2 was found in macrophages and smooth muscle cells (8).

Microarray analysis of messenger RNA in human samples has also been used to study atherosclerosis. In these studies lesion macrophages have been compared to resident macrophages obtained from healthy tissue (9). Coronary artery samples have been dissected from explanted hearts of patients undergoing heart transplantation. Portions of arteries from atherosclerotic lesions and from unaffected portions were separated by dissection under a microscope and RNA was isolated without segregation of various cell types. These RNA samples were used to make inferences about the pathogenesis of atherosclerosis (10). Microarray analysis of DNA methylation has also been used to distinguish samples of atherosclerotic lesion samples from normal healthy blood vessel samples (11). In another study investigators have used microarray analysis of atherosclerotic coronary artery samples compared to controls to define genes whose expression levels different between the samples. The most informative genes identified were TNPO1, RAP1B, ZDHHC17, and PPM1B (12). Another similar study identified a different set of analytes (13). A microarray study of peripheral blood from patients with coronary artery atherosclerosis revealed an RNA species that can be used as a biomarker but the RNA was isolated from whole blood without distinguishing among different cell types or between cells and plasma (14).

Samples of artery from patients with coronary artery atherosclerosis are difficult to obtain from living patients. That likely explains why there are no studies of this type.

### SUMMARY OF INVENTION

The invention is based on the inventor's hypothesis that atherosclerosis could be diagnosed with assays for gene expression of one or more components of the STING pathway that are associated with specific cell-types from the peripheral blood. Therefore, the inventor tested the hypothesis by assessing the expression of various components of the STING pathway in four types of peripheral blood cells from subjects with variable degrees of atherosclerosis. The hypothesis was confirmed by the results of those assessments as shown in the Example.

The invention relates to methods for the development of new diagnostic tests and pharmaceutical agents that are relevant to the treatment of patients with atherosclerosis. A preliminary study that was done by the inventors showed that patients with coronary artery calcification express lower levels of STING phosphorylated on serine 366 in monocytes compared to samples from controls that do not demonstrate coronary artery calcification. (Coronary artery calcification is a risk factor for cardiovascular disease that is associated with coronary artery atherosclerosis.) Because phospho-STING(ser366) regulates STING activity and STING acts to enhance type I interferon production, the finding indicated that calcification of the coronary arteries is associated with decreased phospho-STING(ser366) or decreased regulation of STING activity.

As shown in the Example, a second study confirmed the relationship between decreasing phospho-STING(ser366) and disease initiation (the difference between patients with no coronary artery calcification and patients with the presence of coronary artery calcification and subclinical disease) and disease progression (the difference between patients with coronary artery calcification and subclinical disease and patients with overt coronary artery disease) and expanded the findings to indicate that phospho-STING(ser366) is decreased with disease initiation in CD4⁺ T cells, CD8⁺ T cells, B lymphocytes and monocytes and with disease progression in B lymphocytes and monocytes. Furthermore, phospho-TBK1(ser172) (TANK-binding kinase 1) was found to be increased in all mononuclear cell-types with disease initiation and progression. In monocytes IRF3 was found to be decreased with disease progression. In both T cell subsets, phospho-RelA(ser536) was increased with disease progression. In monocytes and B cells, phospho-Akt(thr308) was decreased with disease progression. In monocytes STING was decreased with disease progression. In CD8⁺ T lymphocytes, B cells, and monocytes, BDNF (brain derived neurotrophic factor) was decreased with disease progression. In all mononuclear cell-types, NLRP3 (NACHT, LRR, and PYD domains-containing protein 3) was decreased with disease progression. In monocytes MyD88 was decreased with disease progression.

These findings now provide methods for developing diagnostic tests and therapeutic agents to manage and treat patients with coronary artery atherosclerosis or atherosclerosis of other arteries. Analysis of the molecules in specific cell types provides new diagnostic tests that can be used to manage patients with atherosclerotic vascular disease and, based on the inhibition or enhancement of the molecules identified, new biomarkers for developing pharmaceutical agents.

In a broad aspect, the invention is directed to detecting gene expression of one or more components of the STING pathway in a sample of nucleated peripheral blood cells. In one embodiment, the nucleated peripheral blood cells are mononuclear cells. In one embodiment, the mononuclear cells include B cells, T cells, natural killer cells and monocytes. In one embodiment, the T cells are CD8⁺ or CD4⁺ T cells. In one embodiment, the component of the STING pathway is selected from the group consisting of STING, phospho-STING, Akt, phospho-Akt, IRF3, phospho-IRF3, TBK1, phospho-TBK1, ULK1, phospho-ULK1, cGAS, NLRP3, RelA, phospho-RelA, BDNF, VEGF, MyD88, Trex1, AIM2, Caspase1, SURF4, STEEP1, STIM1, HER2, Stat6 and Src. In one embodiment, the peripheral blood cells are derived from a subject having or at risk for having atherosclerosis. In one embodiment, the sample of peripheral blood cells is derived from a normal control subject that does not have, or is not at risk for having, atherosclerosis.

The invention includes the following numbered embodiments.

1. A method to test a subject for atherosclerosis, the method comprising obtaining peripheral blood cells from said subject and detecting gene expression of one or more components of the STING pathway in those cells.

2. The method of 1, wherein the peripheral blood cells are mononuclear cells.

3. The method of 1-2, wherein gene expression of one or more components of the STING pathway is in specific mononuclear cell types.

4. The method of 2-3, wherein the specific mononuclear cell-types are selected from the group consisting of B lymphocytes, T lymphocytes, monocytes, and natural killer cells.

5. The method of 1, wherein the peripheral blood cells are selected from the group consisting of B lymphocytes, T lymphocytes, monocytes and natural killer (NK) cells.

6. The method of 5, wherein the T lymphocytes are CD8⁺ T lymphocytes or CD4⁺ T lymphocytes.

7. The method of 1-6, wherein the component is selected from the group consisting of STING, phospho-STING, Akt, phospho-Akt, IRF3, phospho-IRF3, TBK1, phospho-TBK1, ULK1, phospho-ULK1, cGAS, NLRP3, RelA, phospho-RelA, BDNF, VEGF, MyD88, Trex1, AIM2, Caspase1, SURF4, STEEP1, STIM1, HER2, Stat6 and Src.

8. The method of 1-7, wherein the detection assay is selected from the group consisting of expression of STING in monocytes; phospho-STING in monocytes, phospho-STING in B lymphocytes, phospho-STING in CD4⁺ T lymphocytes, phospho-STING in CD8⁺ T lymphocytes; IRF3 in monocytes, IRF3 in CD4⁺ T lymphocytes, IRF3 in CD8⁺ T lymphocytes; BDNF in monocytes, BDNF in B lymphocytes, BDNF in CD4⁺ T lymphocytes, BDNF in CD8⁺ T lymphocytes; phospho-RelA in CD4⁺ T lymphocytes, phospho-RelA in CD8⁺ T lymphocytes; phospho-TBK1 in monocytes, phospho-TBK1 in B lymphocytes, phospho-TBK1 in CD4⁺ T lymphocytes, phospho-TBK1 in CD8⁺ T lymphocytes; phospho-Akt in monocytes, phospho-Akt in B lymphocytes; MyD88 in monocytes; and NLRP3 in monocytes, NLRP3 in B lymphocytes, NLRP3 in CD4⁺ T lymphocytes, and NLRP3 in CD8⁺ T lymphocytes.

9. The method of 8, wherein the detection assay is for STING in monocytes.

10. The method of 8, wherein the detection assay is for phospho-STING in monocytes.

11. The method of 8, wherein the detection assay is for phospho-STING in B lymphocytes.

12. The method of 8, wherein the detection assay is for phospho-STING in CD4⁺ T lymphocytes.

13. The method of 8, wherein the detection assay is for phospho-STING in CD8⁺ T lymphocytes.

14. The method of 8, wherein the detection assay is for phospho-Akt in monocytes.

15. The method of 8, wherein the detection assay is for phospho-Akt in B lymphocytes.

16. The method of 8, wherein the detection assay is for IRF3 in monocytes.

17. The method of 8, wherein the detection assay is for IRF3 in CD4⁺ T lymphocytes.

18. The method of 8, wherein the detection assay is for IRF3 in CD8⁺ T lymphocytes.

19. The method of 8, wherein the detection assay is for phospho-TBK1 in monocytes.

20. The method of 8, wherein the detection assay is for phospho-TBK1 in B lymphocytes.

21. The method of 8, wherein the detection assay is for phospho-TBK1 in CD4⁺ T lymphocytes.

22. The method of 8, wherein the detection assay is for phospho-TBK1 in CD8⁺ T lymphocytes.

23. The method of 8, wherein the detection assay is for NLRP3 in monocytes.

24. The method of 8, wherein the detection assay is for NLRP3 in B lymphocytes.

25. The method of 8, wherein the detection assay is for NLRP3 in CD4⁺ T lymphocytes.

26. The method of 8, wherein the detection assay is for NLRP3 in CD8⁺ T lymphocytes.

27. The method of 8, wherein the detection assay is for phospho-RelA in CD4⁺ T lymphocytes.

28. The method of 8, wherein the detection assay is for phospho-RelA in CD8⁺ T lymphocytes.

29. The method of 8, wherein the detection assay is for BDNF in monocytes.

30. The method of 8, wherein the detection assay is for BDNF in B lymphocytes.

31. The method of 8, wherein the detection assay is for BDNF in CD4⁺ T lymphocytes.

32. The method of 8, wherein the detection assay is for BDNF in CD8⁺ T lymphocytes.

33. The method of 8, wherein the detection assay is for MyD88 in monocytes.

34. The method of 1-33, wherein the atherosclerosis is selected from the group consisting of coronary artery disease, carotid artery disease, aortic artery disease, peripheral artery disease, such as, arteries of the upper and lower extremities, and disease of renal arteries.

35. The method of 1-34, wherein the detection assay is selected from the group consisting of flow cytometry, mass cytometry, Western analysis, mass spectrometry, immunoassays, cell-specific enzyme-linked immunosorbent assays, immunohistochemistry, and immunoblotting.

36. The method of 1-35, wherein the detection assay is flow cytometric.

37. The method of 1-36, wherein the detection assay comprises detecting a co-stain on individual peripheral blood cells wherein the co-stain is for (I) a cell lineage marker for the peripheral blood cells and (II) one or more components of the STING pathway.

38. The method of 37, wherein the cell lineage marker is a marker selected from the group consisting of a marker of B lymphocytes, T lymphocytes, monocytes and natural killer (NK) cells.

39. The method of 37, wherein the co-stain is on individual cells.

40. The method of 38, wherein the detection assay comprises detecting a co-stain on individual cells wherein the co-stain is for (I) a cell lineage marker for peripheral blood cells and (II) one or more components of the STING pathway, selected from the group consisting of STING in monocytes; phospho-STING in monocytes, phospho-STING in B lymphocytes, phospho-STING in CD4⁺ T lymphocytes, phospho-STING in CD8⁺ T lymphocytes; phospho-Akt in monocytes, phospho-Akt in B lymphocytes; IRF3 in monocytes, IRF3 in CD4⁺ T lymphocytes, IRF3 in CD8⁺ T lymphocytes; phospho-TBK1 in monocytes, phospho-TBK1 in B lymphocytes, phospho-TBK1 in CD4⁺ T lymphocytes, phospho-TBK1 in CD8⁺ T lymphocytes; NLRP3 in monocytes, NLRP3 in B lymphocytes, NLRP3 in CD4⁺ T lymphocytes, NLRP3 in CD8⁺ T lymphocytes; phospho-RelA in CD4⁺ T lymphocytes, phospho-RelA in CD8⁺ T lymphocytes; BDNF in monocytes, BDNF in B lymphocytes, BDNF in CD4⁺ T lymphocytes, BDNF in CD8⁺ T lymphocytes; and MyD88 in monocytes.

41. A method for co-staining a peripheral blood cell for a cell-lineage marker of the peripheral blood cell and one or more components of the STING pathway on the peripheral blood cell. The peripheral blood cell can be a mononuclear cell. The various types of mononuclear cells are found throughout this application. The one or more STING pathway components are also disclosed throughout this application. In this method, the signal for one or both of the cell-lineage markers and one or more STING pathway components is amplified. In one embodiment, only the marker for the STING pathway component is amplified. Amplification includes enzyme-catalyzed reporter technology as disclosed in this application. An enzyme can be conjugated with a primary antibody for the cell-lineage marker and/or the STING pathway component. One or more reporters may be linked to the enzyme. The reporter can be labeled with a fluorochrome. In a specific embodiment, the enzyme is a peroxidase. In another specific embodiment, the reporter that is labeled with the fluorochrome is tyramide. In a specific embodiment, the fluorochrome can be fluorescein. In a specific embodiment, the peroxidase is horseradish peroxidase.

42. The method of 1-41, wherein the detection assay is performed on a heterogeneous population of peripheral blood cells.

43. The method of 1-41, wherein the detection assay is performed on a homogeneous population of peripheral blood cells.

44. A method to test a compound for its ability to modulate one or more components of the STING pathway in peripheral blood cells. In one embodiment, the peripheral blood cells are derived from a subject having or at risk for having atherosclerosis. In one embodiment, modulation is tested in vivo in subjects having or at risk for having atherosclerosis. The types of peripheral blood cells to which the method applies are described throughout this application. The one or more components of the STING pathway are also described throughout this application.

45. A method to test an agent for the potential to treat atherosclerosis, the method comprising applying the agent to peripheral blood cells and performing an assay to detect modulation of gene expression of one or more components of the STING pathway by the application of the agent. In one embodiment, an increase in gene expression is measured. In one embodiment a decrease in gene expression is measured. The increase and decrease are relative to an appropriate baseline control, which could be a normal subject population, i.e., a population not having or not at risk for having atherosclerosis. Alternatively, the increase or decrease could be relative to the level of gene expression in a particular subject prior to being treated. In one embodiment, the method is performed in vitro with peripheral blood cells from a normal population. In one embodiment, the method is performed in vitro with peripheral blood cells from a subject having or at risk for having atherosclerosis. In one embodiment, the method is applied in vivo to a subject having or at risk for having atherosclerosis. Peripheral blood cells to which the method applies are described throughout this application. Components of the STING pathway to which this method applies are described throughout this application. Subjects with various types of atherosclerosis or at risk for atherosclerosis are also described in this application.

46. A method for testing a compound for its effects on atherosclerosis, the method comprising administering a compound to a subject having or at risk for having atherosclerosis and assessing the effect of the compound on the development, progression or severity of the disease, the compound being an inducer or inhibitor of one or more components of the STING pathway.

47. The method of 46, wherein the atherosclerosis is selected from the group consisting of coronary artery disease, carotid artery disease, aortic artery disease, peripheral artery disease, such as, arteries of the upper and lower extremities, and disease of renal arteries.

48. The method of 1-47, wherein the component is selected from the group consisting of STING, phospho-STING, Akt, phospho-Akt, IRF3, phospho-IRF3, TBK1, phospho-TBK1, ULK1, phospho-ULK1, cGAS, NLRP3, RelA, phospho-RelA, BDNF, VEGF, MyD88, Trex1, AIM2, Caspase1, SURF4, STEEP1, STIM1, HER2, Stat6 and Src.

49. A method to treat a subject having or at risk for having atherosclerosis, the method comprising administering an inducer or inhibitor of one or more components of the STING pathway in a therapeutically effective amount and for a sufficient time to treat the subject.

50. The method of 49, wherein the atherosclerosis is selected from the group consisting of coronary artery disease, carotid artery disease, aortic artery disease, peripheral artery disease, such as, arteries of the upper and lower extremities, and disease of renal arteries.

51. The method of 49-50, wherein the component is selected from the group consisting of STING, phospho-STING, Akt, phospho-Akt, IRF3, phospho-IRF3, TBK1, phospho-TBK1, ULK1, phospho-ULK1, cGAS, NLRP3, RelA, phospho-RelA, BDNF, VEGF, MyD88, Trex1, AIM2, Caspase1, SURF4, STEEP1, STIM1, HER2, Stat6 and Src.

52. A composition comprising peripheral blood cells, wherein individual peripheral blood cells are co-stained for a cell-lineage marker for the peripheral blood cell and for a marker for one or more components of the STING pathway.

53. The composition of 52 wherein the peripheral blood cell is a mononuclear cell.

54. The composition of 53 wherein the mononuclear cell is selected from the group consisting of monocytes, B cells, and T cells.

55. The composition of 54 wherein the mononuclear cell is a monocyte.

56. The composition of 54 wherein the mononuclear cell is a B cell.

57. The composition of 54 wherein the mononuclear cell is a T cell.

58. The composition of 57 wherein the T cell is a CD8⁺ T cell or CD4⁺ T cell.

59. The composition of 52 wherein the cell-lineage marker for the peripheral blood cell is a marker of peripheral blood mononuclear cells.

60. The composition of 59 wherein the marker is for B cells.

61. The composition of 60 wherein the marker is for T cells.

62. The composition of 61 wherein the marker is for CD8⁺ T cells or CD4⁺ T cells.

63. The composition of 59 wherein the marker is for monocytes.

64. The composition of 52-63 wherein the marker for a component of the STING pathway is selected from the group consisting of STING, phospho-STING, Akt, phospho-Akt, IRF3, phospho-IRF3, TBK1, phospho-TBK1, ULK1, phospho-ULK1, cGAS, NLRP3, RelA, phospho-RelA, BDNF, VEGF, MyD88, Trex1, AIM2, Caspase1, SURF4, STEEP1, STIM1, HER2, Stat6 and Src.

65. The composition of 52-64 wherein the co-stain comprises an antibody.

66. The composition of 52-65 wherein cell-lineage markers for B cells include CD19 or CD20; cell-lineage markers for CD4⁺ T cells include CD3 and/or CD4; cell-lineage markers for CD8⁺ T cells include CD3 and/or CD8; cell-lineage markers for natural killer (NK) cells include CD56 and/or CD16; cell-lineage markers for monocytes include CD14 and/or CD4.

67. The composition of 52-66 in which the detection signal for the cell-lineage marker and/or the STING pathway component marker is amplified.

68. A composition comprising peripheral blood cells in which individual peripheral blood cells are co-stained with an antibody for a cell-lineage marker and an antibody for one or more components of the STING pathway, the composition further comprising an amplification medium.

69. The composition of 68 wherein the antibody is bound to a peroxidase.

70. The composition of 68-69 wherein the medium further contains tyramide.

71. The composition of 68-70 wherein the signal is amplified by means of tyramide.

72. A kit containing antibodies to one or more cell-lineage markers on peripheral blood cells, antibodies to one or more STING pathway components, an amplification medium and a wash medium. The kit can further contain a cell-fixation reagent and a cell-permeabilization reagent. The one or more peripheral blood cells include mononuclear cells. The mononuclear cells include B lymphocytes, T lymphocytes, monocytes and natural killer cells. The one or more STING pathway components include STING, phospho-STING, Akt, phospho-Akt, IRF3, phospho-IRF3, TBK1, phospho-TBK1, ULK1, phospho-ULK1, cGAS, NLRP3, RelA, phospho-RelA, BDNF, VEGF, MyD88, Trex1, AIM2, Caspase1, SURF4, STEEP1, STIM1, HER2, Stat6 and Src.

Kit components can allow for direct staining, indirect staining without amplification and indirect staining with amplification as described in the Detailed Description.

### BRIEF DESCRIPTION OF THE DRAWINGS

FIG. 1 shows representative analyte expression flow cytometric histograms for monocytes from a single donor. All the analytes assessed are intracellular molecules; consequently, the cells were fixed and permeabilized prior to staining. The expression levels of the analytes were enhanced with CellPrint™.

FIG. 2 shows the expression level of phospho-STING in monocytes and IRF3 in B lymphocytes in samples from patients with calcified atherosclerosis (coronary artery calcium score, CAC score, greater than 5) compared to samples from patients without calcified atherosclerosis (CAC score=0). The p values for t tests between the patients with and without calcified atherosclerosis is shown above the histograms.

FIG. 3 shows the relationship of phospho-STING levels in monocytes and CAC score. Pearson correlation coefficient (r) and the associated p value is shown.

FIG. 4 shows IRF3 expression levels in B lymphocytes from patients without calcified atherosclerosis (CAC score=0) and patients with mild subclinical disease (CAC>5/<200). The p value for t tests between the patients with mild, subclinical atherosclerosis and without calcified atherosclerosis is shown above the histograms.

FIG. 5 shows phospho-STING expression levels in monocytes and phospho-Akt expression levels in B cells between patients with mild subclinical coronary artery calcification (CAC score>51<200) and patients with moderate subclinical disease (CAC score>200). The p value for t tests between the patients with mild, subclinical atherosclerosis and patients with moderate, subclinical atherosclerosis is shown above the histograms.

FIG. 6 shows the results for cell-type specific phospho-RelA expression. Comparisons are made between expression levels of patients with no coronary artery disease (NC), patients with subclinical coronary artery disease (SC; defined as positive coronary artery calcification), and patients with established coronary artery disease (CD; defined clinically and by coronary artery evaluation). The comparisons were made by ANOVA and the p values shown have been corrected by Tukey HSD.

FIG. 7 shows the results for cell-type specific IRF3 expression.

FIG. 8 shows the results for cell-type specific phospho-TBK1.

FIG. 9 shows the results for cell-type specific phospho-Akt expression.

FIG. 10 shows results for cell-type specific BDNF expression.

FIG. 11 shows results for cell-type specific STING expression.

FIG. 12 shows results for cell-type specific phospho-STING expression.

FIG. 13 shows the results for cell-type specific NLRP3 expression.

FIG. 14 shows results for cell-type specific MyD88 expression.

FIG. 15 shows how the expression level of phospho-STING in B lymphocytes can be used to distinguish patients with no coronary artery disease (CAD) from those patients with subclinical coronary artery disease (CAD). The logistic regression is shown with n=63 and p<0.001.

FIG. 16 shows receiver operator characteristics (ROC) analysis of B cell phospho-STING stratifying patients with subclinical coronary artery disease (CAD) from those with none. The area under the curve is 0.85 with a 95% confidence interval of 0.75-0.94 and a p value<0.001.

FIG. 17 shows cell-type specific expression levels (STING levels in monocytes and phospho-TBK1 levels in CD4⁺ T cells) in patients with subclinical coronary artery disease (CAD) and patients with established coronary artery disease (CAD). The logistic regression is shown with n=72 and p<0.001. The p value for the inclusion of monocyte STING in the model is 0.001. The p value for the inclusion of CD4⁺ T cell phospho-TBK1 in the model is <0.001.

FIGS. 18A THROUGH 18E show ROC analysis for individual factors in patients with subclinical coronary artery disease (CAD) and patients with established coronary artery disease (CAD). FIG. 18A shows a ROC (receiver operating characteristic) analysis for phospho-RelA expression levels in CD4⁺ T cells that distinguishes patients with subclinical CAD from patients with clinical CAD. FIG. 18B shows a ROC analysis for STING expression levels in monocytes that distinguishes patients with subclinical CAD from patients with clinical CAD. FIG. 18C shows a ROC analysis for phospho-TBK1 expression levels in CD4⁺ T cells that distinguishes patients with subclinical CAD from patients with clinical CAD. FIG. 18D shows a ROC analysis for BDNF expression levels in monocytes that distinguishes patients with subclinical CAD from patients with clinical CAD. FIG. 18E shows a ROC analysis for phospho-Akt expression levels in monocytes that distinguishes patients with subclinical CAD from patients with clinical CAD.

FIG. 19 shows an of the tyramide signal amplification system.

### DETAILED DESCRIPTION OF THE INVENTION

### Definitions

“STING” is understood to refer to Stimulator of Interferon Gene, a transmembrane protein found in the endoplasmic reticulum and Golgi apparatus, that binds cyclic dinucleotides and subsequently stimulates interferon production by activating TANK-binding kinase 1 and interferon regulatory factor 3. STING is encoded by a gene having, in humans, the sequence shown in NCBI Reference Sequence: MF622062.1. The url follows: https://www.ncbi.nlm.nih.gov/nuccore/MF622062.1, incorporated by reference for the sequence. This gene is also known, like most other genes, to contain polymorphisms that still allow the gene to maintain the function. The gene also includes, for non-human uses, such as veterinary uses, orthologs from other mammals. These include companion animals, farm animals and sport animals, for example, felines, canines, bovines, equines, porcines, ovines, etc.

“Phospho-STING” is understood to refer to Stimulator of Interferon Gene, that is phosphorylated on the amino acid serine at position 366, encoded by a gene having, in humans, the sequence shown in NCBI Reference Sequence: MF622062.1. The url follows: https://www.ncbi.nlm.nih.gov/nuccore/MF622062.1, incorporated by reference for the sequence. Phosphorylation of STING at serine position 366 is known to effect regulatory consequences on the activity of the protein. This gene is also known, like most other genes, to contain polymorphisms that still allow the gene to maintain the function. The gene also includes, for non-human uses, such as veterinary uses, orthologs from other mammals. These include companion animals, farm animals and sport animals, for example, felines, canines, bovines, equines, porcines, ovines, etc.

“Phospho-Akt” is understood to refer to a serine/threonine-specific protein kinase, also known as protein kinase B, that is phosphorylated on the amino acid threonine at position 308, encoded by a gene having, in humans, the sequence shown in NCBI Reference Sequence: NM 005163.2. The url follows: https://www.ncbi.nlm.nih.gov/nuccore/NM_005163.2, incorporated by reference for the sequence. There is also an Akt2 which is closely related to Akt1. It has NCBI Reference Sequence: NM 001243027.1. The url follows: https://www.ncbi.nlm.nih.gov/nuccore/NM_001626.6, incorporated by reference for the sequence. The antibodies used in the Examples detect both Akt1 and Akt2 phosphorylations. There are variants of both Akt1 and Akt2. This gene is also known, like most other genes, to contain polymorphisms that still allow the gene to maintain the function. The gene also includes, for non-human uses, such as veterinary uses, orthologs from other mammals. These include companion animals, farm animals and sport animals, for example, felines, canines, bovines, equines, porcines, ovines, etc.

“IRF3” is understood to refer to interferon regulatory factor 3, encoded by a gene having, in humans, the sequence shown in NCBI Reference Sequence: NG_031810.1. The url follows: https://www.ncbi.nlm.nih.gov/nuccore/NG_031810.1, incorporated by reference for the sequence. IRF3 is known to act as a transcription factor, which in a complex with cyclic adenosine monophosphate response element binding protein binding protein acts to active the transcription of type I interferons. This gene is also known, like most other genes, to contain polymorphisms that still allow the gene to maintain the function. The gene also includes, for non-human uses, such as veterinary uses, orthologs from other mammals. These include companion animals, farm animals and sport animals, for example, felines, canines, bovines, equines, porcines, ovines, etc.

“Phospho-IRF3” is understood to refer to interferon regulatory factor 3, that is phosphorylated on the amino acid serine at position 396, encoded by a gene having, in humans, the sequence shown in NCBI Reference Sequence: NG_031810.1. The url follows: https://www.ncbi.nlm.nih.gov/nuccore/NG_031810.1, incorporated by reference for the sequence. IRF3 is known to act as a transcription factor, which in a complex with cyclic adenosine monophosphate response element binding protein binding protein acts to active the transcription of type I interferons. Phosphorylation of IRF3 at this position is known to mediate a regulatory control of the activity of the protein. This gene is also known, like most other genes, to contain polymorphisms that still allow the gene to maintain the function. The gene also includes, for non-human uses, such as veterinary uses, orthologs from other mammals. These include companion animals, farm animals and sport animals, for example, felines, canines, bovines, equines, porcines, ovines, etc.

“TBK1” is understood to refer to TANK binding kinase 1, with TANK referring to Traf family member-associated NF-kappa-B activator, encoded by a gene having, in humans, the sequence shown in NCBI Reference Sequence: NM_013254.4. The url follows: https://www.ncbi.nlm.nih.gov/nuccore/NM_013254.4, incorporated by reference for the sequence. TBK1 is known to act as a kinase that phosphorylates RelA as well as IRF3 leading to inflammatory responses. This gene is also known, like most other genes, to contain polymorphisms that still allow the gene to maintain the function. The gene also includes, for non-human uses, such as veterinary uses, orthologs from other mammals. These include companion animals, farm animals and sport animals, for example, felines, canines, bovines, equines, porcines, ovines, etc.

“Phospho-TBK1” is understood to refer to TANK binding kinase 1, with TANK referring to Traf family member-associated NF-kappa-B activator, that is phosphorylated on the amino acid serine at position 172, encoded by a gene having, in humans, the sequence shown in NCBI Reference Sequence: NM_013254.4. The url follows: https://www.ncbi.nlm.nih.gov/nuccore/NM_013254.4, incorporated by reference for the sequence. TBK1 is known to act as a kinase that phosphorylates RelA that stimulates an inflammatory response as well as IRF3 that stimulates the production type I interferons. This gene is also known, like most other genes, to contain polymorphisms that still allow the gene to maintain the function. The gene also includes, for non-human uses, such as veterinary uses, orthologs from other mammals. These include companion animals, farm animals and sport animals, for example, felines, canines, bovines, equines, porcines, ovines, etc.

“ULK1” is understood to refer to unc-51 like autophagy activating kinase 1, encoded by a gene having, in humans, the sequence shown in NCBI Reference Sequence: NM_003565.4. The url follows: https://www.ncbi.nlm.nih.gov/nuccore/NM_003565.4, incorporated by reference for the sequence. ULK1 is a kinase important to the autophagy function of cells and known to phosphorylate STING. This gene is also known, like most other genes, to contain polymorphisms that still allow the gene to maintain the function. The gene also includes, for non-human uses, such as veterinary uses, orthologs from other mammals. These include companion animals, farm animals and sport animals, for example, felines, canines, bovines, equines, porcines, ovines, etc.

“Phospho-ULK1” is understood to refer to unc-51 like autophagy activating kinase 1, that is phosphorylated on the amino acid serine at position 757, encoded by a gene having, in humans, the sequence shown in NCBI Reference Sequence: NM_003565.4. The url follows: https://www.ncbi.nlm.nih.gov/nuccore/NM_003565.4, incorporated by reference for the sequence. ULK1 is a kinase important to the autophagy function of cells and known to phosphorylate STING. This gene is also known, like most other genes, to contain polymorphisms that still allow the gene to maintain the function. The gene also includes, for non-human uses, such as veterinary uses, orthologs from other mammals. These include companion animals, farm animals and sport animals, for example, felines, canines, bovines, equines, porcines, ovines, etc.

“cGAS” is understood to refer to cyclic GMP-AMP synthase, encoded by a gene having, in humans, the sequence shown in NCBI Reference Sequence: NM_138441.3. The url follows: https://www.ncbi.nlm.nih.gov/nuccore/NM_138441.3, incorporated by reference for the sequence. cGAS is known to act as a protein that binds double-stranded DNA and consequently produces cyclic-GMP-cyclic-AMP, which binds to and activates STING. This gene is also known, like most other genes, to contain polymorphisms that still allow the gene to maintain the function. The gene also includes, for non-human uses, such as veterinary uses, orthologs from other mammals. These include companion animals, farm animals and sport animals, for example, felines, canines, bovines, equines, porcines, ovines, etc.

“NLRP3” is understood to refer to NACHT, LRR and PYD domains-containing protein 3, encoded by a gene having, in humans, the sequence shown in NCBI Reference Sequence: NG_007509.2. The url follows: https://www.ncbi.nlm.nih.gov/nuccore/NG_007509.2, incorporated by reference for the sequence. NLRP3 functions as an upstream regulator of NFkB signaling. It is also part of a caspase-1 activating complex which results in the production of the inflammatory cytokine IL1b. This gene is also known, like most other genes, to contain polymorphisms that still allow the gene to maintain the function. The gene also includes, for non-human uses, such as veterinary uses, orthologs from other mammals. These include companion animals, farm animals and sport animals, for example, felines, canines, bovines, equines, porcines, ovines, etc.

“RelA” is understood to refer to a regulatory component of the NF-kappa-B transcription factor, encoded by a gene having, in humans, the sequence shown in NCBI Reference Sequence: NG_029971.1. The url follows: https://www.ncbi.nlm.nih.gov/nuccore/NG_029971.1, incorporated by reference for the sequence. RelA, also known as p65, is known to act as part of a transcription factor complex that mediates the transcription of genes involved in inflammatory and immune responses. This gene is also known, like most other genes, to contain polymorphisms that still allow the gene to maintain the function. The gene also includes, for non-human uses, such as veterinary uses, orthologs from other mammals. These include companion animals, farm animals and sport animals, for example, felines, canines, bovines, equines, porcines, ovines, etc.

“Phospho-RelA” is understood to refer to a regulatory component of the NF-kappa-B transcription factor, that is phosphorylated on the amino acid serine at position 536, encoded by a gene having, in humans, the sequence shown in NCBI Reference Sequence: NG_029971.1. The url follows: https://www.ncbi.nlm.nih.gov/nuccore/NG_029971.1, incorporated by reference for the sequence. RelA, also known as p65, is known to act as part of a transcription factor complex that mediates the transcription of genes involved in inflammatory and immune responses. Phosphorylation of RelA regulates the activity of the protein to participate in the induction of gene transcription. This gene is also known, like most other genes, to contain polymorphisms that still allow the gene to maintain the function. The gene also includes, for non-human uses, such as veterinary uses, orthologs from other mammals. These include companion animals, farm animals and sport animals, for example, felines, canines, bovines, equines, porcines, ovines, etc.

“BDNF” is understood to refer to brain-derived neurotrophic factor, encoded by a gene having, in humans, the sequence shown in GenBank Reference number: X91251.1. The url follows: https://www.ncbi.nlm.nih.gov/nuccore/X91251.1, incorporated by reference for the sequence. BDNF is a member of the neurotrophin family of growth factors, acting on neurons to support their survival. This gene is also known, like most other genes, to contain polymorphisms that still allow the gene to maintain the function. The gene also includes, for non-human uses, such as veterinary uses, orthologs from other mammals. These include companion animals, farm animals and sport animals, for example, felines, canines, bovines, equines, porcines, ovines, etc.

“VEGF” is understood to refer to as vascular endothelial growth factor, encoded by a gene having, in humans, the sequence shown in GenBank Reference Sequence: AF024710. The url follows: https://www.ncbi.nlm.nih.gov/nuccore/AF024710, incorporated by reference for the sequence. VEGF is a protein that stimulates the formation of blood vessels. This gene is also known, like most other genes, to contain polymorphisms that still allow the gene to maintain the function. The gene also includes, for non-human uses, such as veterinary uses, orthologs from other mammals. These include companion animals, farm animals and sport animals, for example, felines, canines, bovines, equines, porcines, ovines, etc.

“MyD88” is understood to refer to myeloid differentiation primary response 88, encoded by a gene having, in humans, the sequence shown in GenBank Reference Sequence: U70451.1. The url follows: https://www.ncbi.nlm.nih.gov/nuccore/U70451.1, incorporated by reference for the sequence. MyD88 is an adapter protein involved in translating signals via toll and toll-like receptors into transcriptional events. This gene is also known, like most other genes, to contain polymorphisms that still allow the gene to maintain the function. The gene also includes, for non-human uses, such as veterinary uses, orthologs from other mammals. These include companion animals, farm animals and sport animals, for example, felines, canines, bovines, equines, porcines, ovines, etc.

“Trex1” is understood to refer to three prime repair exonuclease 1, encoded by a gene having, in humans, the sequence shown in GenBank Reference Sequence: AF319566.1. The url follows: https://www.ncbi.nlm.nih.gov/nuccore/AF319566.1, incorporated by reference for the sequence. Trex1 is a major DNA exonuclease in human cells. It is known to degrade cytoplasmic DNA which decreases cGAS activation. This gene is also known, like most other genes, to contain polymorphisms that still allow the gene to maintain the function. The gene also includes, for non-human uses, such as veterinary uses, orthologs from other mammals. These include companion animals, farm animals and sport animals, for example, felines, canines, bovines, equines, porcines, ovines, etc.

“AIM2” is understood to refer to absent in melanoma 2, encoded by a gene having, in humans, the sequence shown in GenBank Sequence: AF024714. The url follows: https://www.ncbi.nlm.nih.gov/nuccore/AF024714.1, incorporated by reference for the sequence. AIM2 is a component of the AIM2 inflammasome. It is a cytoplasmic sensor recognizing the presence of double-stranded DNA. It has been shown to inhibit STING activation. This gene is also known, like most other genes, to contain polymorphisms that still allow the gene to maintain the function. The gene also includes, for non-human uses, such as veterinary uses, orthologs from other mammals. These include companion animals, farm animals and sport animals, for example, felines, canines, bovines, equines, porcines, ovines, etc.

“Caspase1” is understood to refer to a protein, encoded by a gene having, in humans, the sequence shown in NCBI Reference Sequence: NG_029124.2. The url follows: https://www.ncbi.nlm.nih.gov/nuccore/NG_029124.2, incorporated by reference for the sequence. Caspase 1 is a member of the cysteine-aspartic acid protease family, and it is a component of the AIM2 inflammasome, which has been shown to inhibit STING activation. This gene is also known, like most other genes, to contain polymorphisms that still allow the gene to maintain the function. The gene also includes, for non-human uses, such as veterinary uses, orthologs from other mammals. These include companion animals, farm animals and sport animals, for example, felines, canines, bovines, equines, porcines, ovines, etc.

“SURF4” is understood to refer to surfeit locus protein 4, encoded by a gene having, in humans, the sequence shown in GenBank Reference Sequence: CU676307.1. The url follows: https://www.ncbi.nlm.nih.gov/nuccore/CU676407.1, incorporated by reference for the sequence. SURF4 is a protein involved in the transport of STING between the endoplasmic reticulum and the Golgi apparatus. This gene is also known, like most other genes, to contain polymorphisms that still allow the gene to maintain the function. The gene also includes, for non-human uses, such as veterinary uses, orthologs from other mammals. These include companion animals, farm animals and sport animals, for example, felines, canines, bovines, equines, porcines, ovines, etc.

“STEEP1” is understood to refer to STING1 endoplasmic reticulum exit protein 1, encoded by a gene having, in humans, the sequence shown in NCBI Reference Sequence: NG_016378.1. The url follows: https://www.ncbi.nlm.nih.gov/nuccore/NG_016378.1, incorporated by reference for the sequence. STEEP1 regulates the exit of STING from the endoplasmic reticulum. This gene is also known, like most other genes, to contain polymorphisms that still allow the gene to maintain the function. The gene also includes, for non-human uses, such as veterinary uses, orthologs from other mammals. These include companion animals, farm animals and sport animals, for example, felines, canines, bovines, equines, porcines, ovines, etc.

“STIM1” is understood to refer to stromal interaction molecule 1, encoded by a gene having, in humans, the sequence shown in GenBank Reference Sequence: JX014264.1. The url follows: https://www.ncbi.nlm.nih.gov/nuccore/JX014264.1, incorporated by reference for the sequence. STIM1 is an ionized calcium sensor that regulates retention of the STING at the endoplasmic reticulum. This gene is also known, like most other genes, to contain polymorphisms that still allow the gene to maintain the function. The gene also includes, for non-human uses, such as veterinary uses, orthologs from other mammals. These include companion animals, farm animals and sport animals, for example, felines, canines, bovines, equines, porcines, ovines, etc.

“HER2” is understood to refer to human erb-b2 receptor tyrosine kinase 2, encoded by a gene having, in humans, the sequence shown in NCBI Reference Sequence: NG_007503.1. The url follows: https://www.ncbi.nlm.nih.gov/nuccore/NG_007503.1, incorporated by reference for the sequence. HER2 is a member of the epidermal growth factor receptor family shown to negatively regulate STING signaling in conjunction with Akt. This gene is also known, like most other genes, to contain polymorphisms that still allow the gene to maintain the function. The gene also includes, for non-human uses, such as veterinary uses, orthologs from other mammals. These include companion animals, farm animals and sport animals, for example, felines, canines, bovines, equines, porcines, ovines, etc.

“Stat6” is understood to refer to as signal transducer and activator of transcription 6, encoded by a gene having, in humans, the sequence shown in GenBank Reference Sequence: AH006951.2. The url follows: https://www.ncbi.nlm.nih.gov/nuccore/AH006951.2, incorporated by reference for the sequence. Stat6 is a transcription factor activated by various cytokines binding to their receptors. Stat6 is also activated by STING. This gene is also known, like most other genes, to contain polymorphisms that still allow the gene to maintain the function. The gene also includes, for non-human uses, such as veterinary uses, orthologs from other mammals. These include companion animals, farm animals and sport animals, for example, felines, canines, bovines, equines, porcines, ovines, etc.

“Src” is understood to refer to as a proto-oncogene tyrosine-protein kinase, encoded by a gene having, in humans, the sequence shown in NCBI Reference Sequence: NG_023033.1. The url follows: https://www.ncbi.nlm.nih.gov/nuccore/NG_023033.1, incorporated by reference for the sequence. Src promotes phosphorylation of the kinase TBK1 to facilitate type I interferon production. This gene is also known, like most other genes, to contain polymorphisms that still allow the gene to maintain the function. The gene also includes, for non-human uses, such as veterinary uses, orthologs from other mammals. These include companion animals, farm animals and sport animals, for example, felines, canines, bovines, equines, porcines, ovines, etc.

The genes include variants that retain the function of the gene sequence that is disclosed in this application and that, therefore, can be used to practice the methods and compositions that are disclosed in this application.

Unless otherwise defined, all technical and scientific terms used herein have the same meaning as commonly understood by one of ordinary skill in the art to which this invention pertains. In case of conflict, the present specification, including definitions, will control. The terminology as set forth herein is for description of the embodiments only and should not be construed as limiting the application as a whole. Unless otherwise specified, “a,” “an,” “the,” “one or more,” and “at least one” are used interchangeably. Furthermore, as used in the description of the application and the appended claims, the singular forms “a,” “an” and “the” are inclusive of their plural forms, unless contraindicated by the context surrounding such. Furthermore, the recitation of numerical ranges by endpoints includes all of the numbers subsumed within that range (e.g., 1 to 5 includes 1, 1.5, 2, 2.75, 3, 3.80, 4, 5, etc.).

In this application, the use of the terms “atherosclerosis,” “atherosclerotic disease (AD)” are used interchangeably. The application may also refer to “arterial atherosclerotic disease (AAD).”

“Co-administer” means to administer in conjunction with one another, together, coordinately, including simultaneous or sequential administration of two or more agents.

“Comprised of” is a synonym of “comprising.”

“Comprising” means, without other limitation, including the referent, necessarily, without any qualification or exclusion on what else may be included. For example, “a composition comprising x and y” encompasses any composition that contains x and y, no matter what other components may be present in the composition. Likewise, “a method comprising the step of x” encompasses any method in which x is carried out, whether x is the only step in the method or it is only one of the steps, no matter how many other steps there may be and no matter how simple or complex x is in comparison to them. “Comprised of” and similar phrases using words of the root “comprise” are used herein as synonyms of “comprising” and have the same meaning.

“Decrease” or “reduce” means to prevent entirely as well as to lower. With respect to the invention, a “decrease” may refer to a reduction in gene expression relative to the range that is found in a normal population. With respect to a treated population, the terms can refer to a lowered gene expression in the treated subject relative to the level of expression in the subject prior to treatment.

“EAS” and “CellPrint” are trademarked by the inventor. The generic description is enzymatic amplification staining. It is a procedure to amplify the signal in flow cytometric analysis. Patents describing the procedure include U.S. Pat. Nos. 6,280,961, 6,335,173 and 6,828,109.

“Effective amount” generally means an amount which provides the desired effect. For example, an effective amount is an amount sufficient to effectuate a beneficial or desired clinical result. The effective amounts can be provided all at once in a single administration or in fractional amounts that provide the effective amount in several administrations. The precise determination of what would be considered an effective amount may be based on factors individual to each subject, including their size, age, injury, and/or disease or injury being treated, and amount of time since the injury occurred or the disease began. One skilled in the art will be able to determine the effective amount for a given subject based on these considerations which are routine in the art. As used herein, “effective dose” means the same as “effective amount.” With respect to the invention, the “effective amount” also includes that amount which produces a desired reduction or increase in gene expression of a component of the STING pathway. More broadly, it includes that amount that reduces the symptoms of atherosclerosis in the treated subject.

“Effective route” generally means a route which provides for delivery of an agent to a desired compartment, system, or location. For example, an effective route is one through which an agent can be administered to provide at the desired site of action an amount of the agent sufficient to effectuate a beneficial or desired clinical result.

“Includes” is not intended to be limiting.

“Increase” or “increasing” means to induce entirely, where there was no pre-existing effect, as well as to increase the degree. Accordingly, an “increase” can be from essentially zero (-0-) or non-detectible or can be increased over a certain level of existing gene expression. According to the invention, an “increase” in the gene expression of a component of the STING pathway may be relative to that found in the normal range of a normal (non-atherosclerotic) population. “Increasing” could also mean, in a treated patient, raising the level of gene expression of a component of the STING pathway in a subject relative to the level that existed in that subject prior to treatment.

As used herein, the term “elevated” may contain a specific number or specific range that defines the term. Where such number or range is not provided numerically, it is understood that “elevation” would be relative to the number or range in a normal control population (non-atherosclerotic subjects).

“Inducer” refers to a compound that results in enhanced gene expression relative to a control. The control may show no expression, and, therefore, enhancement is any expression of the gene. Or the control may be expression at a certain level, and enhancement, therefore, is expression that is increased beyond that level. Accordingly, the control could be the range of gene expression in a normal (non-atherosclerotic) population. Or the control could be the level of gene expression in the treated subject prior to treatment of the subject.

“Inhibitor” refers to a compound that decreases gene expression relative to a control. Accordingly, the control could be the range of gene expression in a normal (non-atherosclerotic) population. Or the control could be the level of gene expression in the treated subject prior to treatment.

The term “isolated” refers to a cell or cells that is/are not associated with one or more cells or one or more cellular components that are associated with the cell or cells in vivo. An “enriched population” means a relative increase in numbers of a desired cell relative to one or more other cell types in vivo or in primary culture. With respect to the invention, specific peripheral blood cells could be isolated and purified to various degrees including substantially homogeneous. These include the peripheral blood cells that are described in this application.

“Isolated” can indicate that the cells are removed from their natural tissue environment and are present at a higher concentration as compared to the normal tissue environment. Accordingly, an “isolated” cell population may further include cell types in addition to the cells at issue and may include additional tissue components. This also can be expressed in terms of cell doublings, for example. A cell may have undergone 10, 20, 30, 40 or more doublings in vitro or ex vivo so that it is enriched compared to its original numbers in vivo or in its original tissue environment (for example, peripheral blood).

“Substantially homogeneous” (see below) refers to cell preparations where the cell type is of significant purity of at least 50%. The range of homogeneity may, however, be up to and including 100%. Accordingly, the range includes about 50% to 60%, about 60% to 70%, about 70% to 80%, about 80% to 90% and about 90% to 100%. This is distinguished from the term “isolated,” which can refer to levels that are substantially less. However, as used herein, the term “isolated” refers to preparations in which the cells are found in numbers sufficient to allow the detection of a clinically-relevant biological effect, such as, gene expression.

“Pharmaceutically-acceptable carrier” is any pharmaceutically-acceptable medium for administration to a subject. Such a medium may retain isotonicity, cell metabolism, pH, and the like. It is compatible with administration to a subject in vivo, and can be used, therefore, for testing a compound and treatment.

“Reduce” as used herein means to prevent as well as decrease. In the context of treatment, to “reduce” is to both prevent or ameliorate one or more clinical symptoms. A clinical symptom is one (or more) that has or will have, if left untreated, a negative impact on the quality of life (health) of the subject. In the context of testing a compound, “reduce” also would mean to prevent as well as to decrease. In the context of testing a subject, a “reduction” refers to the level of gene expression relative to a control. As indicated above, a control could be a range found in a normal population (non-atherosclerotic) as well as an existing level in a subject prior to treatment of the subject.

“Selecting” a cell can apply to the present invention with respect to testing compounds for drug discovery. Accordingly, a peripheral blood (PB) cell having a certain level of gene expression of a STING component may be selected to test a compound for its effect on the expression level in that cell. So a cell can be selected that has a desired level of gene expression. The cell can be identified (as by assay) and expanded. Or one could create a population that has a higher level of gene expression than the cell that was isolated.

“Subject” means a vertebrate, such as a mammal, such as a human Mammals include, but are not limited to, humans, dogs, cats, horses, cows, and pigs. In the present case, the subject has or is at risk for having atherosclerosis.

“Substantially homogeneous” refers to cell preparations where the cell type is of significant purity of at least 50%. The range of homogeneity may, however, be up to and including 100%. Accordingly, the range includes about 50% to 60%, about 60% to 70%, about 70% to 80%, about 80% to 90% and about 90% to 100%. This is distinguished from the term “isolated,” which can refer to levels that are substantially less. However, as used herein, the term “isolated” refers to preparations in which the cells are found in numbers sufficient to exert a clinically-relevant biological effect.

“Therapeutically effective amount” refers to the amount of an agent determined to produce any therapeutic response in a mammal. For example, effective amounts may prolong the survivability of the patient, and/or inhibit overt clinical symptoms. Treatments that are therapeutically effective within the meaning of the term as used herein, include treatments that improve a subject's quality of life even if they do not improve the disease outcome per se. Such therapeutically effective amounts are readily ascertained by one of ordinary skill in the art. Thus, to “treat” means to deliver such an amount. Thus, treating can prevent or ameliorate any pathological symptoms.

“Treat,” “treating,” or “treatment” are used broadly in relation to the invention and each such term encompasses, among others, preventing, ameliorating, inhibiting, or curing a deficiency, dysfunction, disease, or other deleterious process, including those that interfere with and/or result from a therapy.

Atherosclerosis of the coronary arteries is diagnosed by catheterization and coronary artery angiography, exercise stress tests, and/or noncontrast electrocardiographic-gated cardiac electron beam computerized tomography or multidetector computed tomography.

Atherosclerosis of the carotid arteries is diagnosed by the presence of bruits heard by auscultation, evidence of narrowing of the vessel seen by ultrasound imaging, doppler ultrasound, or computerized tomography angiography, and/or narrowing or blockage seen by cerebral angiography which involves injection of a contrast dye into the carotid arteries.

Atherosclerosis of peripheral arteries is diagnosed by the ankle-brachial index which compares blood pressure in the ankle with blood pressure in your arm before and immediately after exercising on a treadmill, doppler ultrasound, and/or catheter angiography.

Renal artery atherosclerosis is diagnosed by ultrasonography and/or invasive renal artery angiography.

Aortic atherosclerosis is diagnosed by various imaging techniques including transesophageal echocardiography, modified transesophageal echocardiography, epiaortic ultrasound, computed tomography, and/or magnetic resonance imaging.

For the purposes of the invention, subjects “at risk for having” atherosclerosis include subjects with one or more of the following conditions: history of smoking, hypertension (greater than 140 mm Hg systolic and/or greater than 90 mm Hg diastolic), low serum levels of high-density lipoprotein cholesterol (less than 45 milligrams/deciliter), elevated serum levels of low-density lipoprotein cholesterol (greater than 100 milligrams/deciliter), metabolic syndrome, diabetes, obesity (body mass index greater than 30), depression, elevated serum levels of C-reactive protein (greater than 1 milligram/liter), presence of arterial calcium deposits, elevated serum levels of myeloperoxidase (in the fourth quartile or greater than 350 micrograms/liter in serum), or a family history of atherosclerosis.

“History of smoking” is understood to refer to tobacco cigarette smoke consumption by inhalation. It includes any level of consumption equal to or greater than 5 cigarettes per day or the equivalent of other forms of smoke consumption such as cigars. It also includes second-hand smoke exposure. Smoke exposure, by direct or indirect consumption, can be ascertained by urinary cotinine levels.

“Hypertension” is understood to refer to persons with elevated systolic (>140 mm Hg) or diastolic (>90 mm Hg) arterial pressures or persons without elevated arterial pressures who are being treated with anti-hypertensive agents.

“Metabolic syndrome” is understood to refer to persons with at least three of the following conditions: high fasting serum blood glucose, low serum levels of high-density lipoprotein, high serum levels of triglycerides, large waist circumference, and high arterial blood pressure. The term “high” refers to levels that are higher than the range in a normal subject population, which includes subjects that do not have or are not at risk of having atherosclerosis.

“Diabetes” is understood to refer to persons with either type 1 or type 2 diabetes who have elevated fasting blood glucose levels or persons without elevated fasting blood glucose levels who have been treated for diabetes.

“Obesity” is understood to refer to persons with a body mass index of 30 or greater. The body mass index is calculated from the height and weight of a person. Also, the term “obesity” refers to persons with a waist circumference of 35 inches or greater for women or 40 inches or greater for men.

“High-sensitivity C-reactive protein” is understood to refer to the measurement of C-reactive protein in the range from 0.5 to 10 milligram/liter. The term “C-reactive protein” is understood to refer to a protein encoded by a gene having, in humans, the sequence shown in GenBank Reference Sequence: M11725.1. The url follows: https://www.ncbi.nlm.nih.gov/nuccore/M11725.1, incorporated by reference for the sequence. C-reactive protein is a protein whose levels increase in the blood in response to inflammation. This gene is also known, like most other genes, to contain polymorphisms that still allow the gene to maintain the function. The gene also includes, for non-human uses, such as veterinary uses, orthologs from other mammals. These include companion animals, farm animals and sport animals, for example, felines, canines, bovines, equines, porcines, ovines, etc. Elevated serum levels of C-reactive protein refers to levels greater than 1 milligram/liter.

“Arterial calcium deposits” is understood to refer to the presence of calcium in the walls of arteries. Calcium deposits are imaged by either invasive or non-invasive imaging techniques. Calcification is defined as a density equal to or greater than 130 Hounsfield units and an area greater to or equal to 3 pixels. A semiautomated method is used to calculate a calcium score. Any score greater than zero indicates arterial calcium deposits.

“Myeloperoxidase” is understood to refer to a protein encoded by a gene having, in humans, the sequence shown in GenBank Reference Sequence: DQ088846.1. The url follows: https://www.ncbi.nlm.nih.gov/nuccore/DQ088846.1, incorporated by reference for the sequence. Myeloperoxidase is an enzyme that produces hypochlorous acid from hydrogen peroxide and chloride anion. This gene is also known, like most other genes, to contain polymorphisms that still allow the gene to maintain the function. The gene also includes, for non-human uses, such as veterinary uses, orthologs from other mammals. These include companion animals, farm animals and sport animals, for example, felines, canines, bovines, equines, porcines, ovines, etc. Elevated serum myeloperoxidase refers to patients in the fourth quartile or greater than 350 micrograms/liter.

“Low-density lipoprotein” is understood to refer to proteins that transfer lipids through the blood with a density greater than intermediate-density lipoprotein and less than high-density lipoprotein. Low-density lipoprotein transports cholesterol and is typically measured by the amount of associated cholesterol. Elevated levels of serum low-density lipoprotein cholesterol refers to levels greater than 100 milligrams per deciliter.

“High-density lipoprotein” is understood to refer to proteins that transfer lipids through the blood with a density greater than low-density lipoprotein. High-density lipoprotein transports cholesterol and is typically measured by the amount of associated cholesterol. Decreased levels of high-density lipoprotein cholesterol refers to less than 45 milligrams per deciliter.

### Description

There are no cell-associated cell-type-specific molecular expression level biomarkers/diagnostics for the diagnosis of coronary (or other) artery atherosclerosis progression in patients. Without biomarkers/diagnostics the management of these patients is diminished. The invention provides such biomarkers and diagnostic tests to assess the initiation of atherosclerosis and the degree of atherosclerosis progression.

At the same time, there are no pharmaceutical treatments of patients with coronary (or other) artery atherosclerosis based on the activity of immune/inflammatory cells. The dearth of such pharmaceutical agents leaves a potential pathway for interruption of the disease process untapped. The invention thus provides pharmaceutical agents with novel mechanisms of action.

The invention involves the measurement of gene expression in peripheral blood cells to diagnose and manage atherosclerosis patients.

The invention provides a method for providing diagnostic devices and therapeutic agents for the management of patients with atherosclerosis via testing and manipulation of the STING pathway. Inhibition of the STING pathway is useful in treating atherosclerosis patients. Inhibitors of STING have been developed and studied in clinical trials for cancer but not for atherosclerosis (59-61). The STING pathway includes STING, phospho-STING, Akt, phospho-Akt, IRF3, phospho-IRF3, TBK1, phospho-TBK1, ULK1, phospho-ULK1, cGAS, NLRP3, RelA, phospho-RelA, BDNF, VEGF, MyD88, Trex1, AIM2, Caspase1, SURF4, STEEP1, STIM1, HER2, Stat6 and Src.

The genes that are measured to diagnose and manage patients with atherosclerosis are components in the STING pathway.

In one embodiment STING expression levels in peripheral blood cells are measured to diagnose and manage patients with atherosclerosis.

In one embodiment phospho-STING expression levels in peripheral blood cells are measured to diagnose and manage patients with atherosclerosis.

In one embodiment Akt expression levels in peripheral blood cells are measured to diagnose and manage patients with atherosclerosis.

In one embodiment phospho-Akt expression levels in peripheral blood cells are measured to diagnose and manage patients with atherosclerosis.

In one embodiment IRF3 expression levels in peripheral blood cells are measured to diagnose and manage patients with atherosclerosis.

In one embodiment phospho-IRF3 expression levels in peripheral blood cells are measured to diagnose and manage patients with atherosclerosis.

In one embodiment TBK1 expression levels in peripheral blood cells are measured to diagnose and manage patients with atherosclerosis.

In one embodiment phospho-TBK1 expression levels in peripheral blood cells are measured to diagnose and manage patients with atherosclerosis.

In one embodiment ULK1 expression levels in peripheral blood cells are measured to diagnose and manage patients with atherosclerosis.

In one embodiment phospho-ULK1 expression levels in peripheral blood cells are measured to diagnose and manage patients with atherosclerosis.

In one embodiment cGAS expression levels in peripheral blood cells are measured to diagnose and manage patients with atherosclerosis.

In one embodiment NLRP3 expression levels in peripheral blood cells are measured to diagnose and manage patients with atherosclerosis.

In one embodiment RelA expression levels in peripheral blood cells are measured to diagnose and manage patients with atherosclerosis.

In one embodiment phospho-RelA expression levels in peripheral blood cells are measured to diagnose and manage patients with atherosclerosis.

In one embodiment BDNF expression levels in peripheral blood cells are measured to diagnose and manage patients with atherosclerosis.

In one embodiment VEGF expression levels in peripheral blood cells are measured to diagnose and manage patients with atherosclerosis.

In one embodiment MyD88 expression levels in peripheral blood cells are measured to diagnose and manage patients with atherosclerosis.

In one embodiment Trex1 expression levels in peripheral blood cells are measured to diagnose and manage patients with atherosclerosis.

In one embodiment AIM2 expression levels in peripheral blood cells are measured to diagnose and manage patients with atherosclerosis.

In one embodiment Caspase1 expression levels in peripheral blood cells are measured to diagnose and manage patients with atherosclerosis.

In one embodiment SURF4 expression levels in peripheral blood cells are measured to diagnose and manage patients with atherosclerosis.

In one embodiment STEEP1 expression levels in peripheral blood cells are measured to diagnose and manage patients with atherosclerosis.

In one embodiment STIM1 expression levels in peripheral blood cells are measured to diagnose and manage patients with atherosclerosis.

In one embodiment HER2 expression levels in peripheral blood cells are measured to diagnose and manage patients with atherosclerosis.

In one embodiment Stat6 expression levels in peripheral blood cells are measured to diagnose and manage patients with atherosclerosis.

In one embodiment phospho-Stat6 expression levels in peripheral blood cells are measured to diagnose and manage patients with atherosclerosis.

In one embodiment Src expression levels in peripheral blood cells are measured to diagnose and manage patients with atherosclerosis.

In one embodiment the atherosclerotic process involves the coronary arteries.

In one embodiment the atherosclerotic process involves the carotid arteries.

In one embodiment the atherosclerotic process involves arteries in the lower extremities.

In one embodiment the atherosclerotic process involves the aorta.

In one embodiment the atherosclerotic process involves a renal artery.

In one embodiment the atherosclerotic process involves arteries in the upper extremities.

In one embodiment gene expression is measured in one or more peripheral blood mononuclear cells, including, CD8⁺ T cells, natural killer cells, γ/δ T cells, NKT cells, CD4⁺ T cells, monocytes, or B cells.

The activity of an inhibitor or inducer to effectively treat a subject with atherosclerosis can be measured by a quantitative assay to detect the effect of the inhibitor or inducer on the gene expression of one or more STING pathway components in peripheral blood cells.

In one embodiment STING expression levels in specific cell-types from the peripheral blood can be used to develop drugs to treat patients with atherosclerosis.

In one embodiment phosphorylated STING expression levels in specific cell-types from the peripheral blood can be used to develop drugs to treat patients with atherosclerosis.

In one embodiment Akt expression levels in specific cell-types from the peripheral blood can be used to develop drugs to treat patients with atherosclerosis.

In one embodiment phosphorylated Akt expression levels in specific cell-types from the peripheral blood can be used to develop drugs to treat patients with atherosclerosis.

In one embodiment IRF3 expression levels in specific cell-types from the peripheral blood can be used to develop drugs to treat patients with atherosclerosis.

In one embodiment phosphorylated IRF3 expression levels in specific cell-types from the peripheral blood can be used to develop drugs to treat patients with atherosclerosis.

In one embodiment TBK1 expression levels in specific cell-types from the peripheral blood can be used to develop drugs to treat patients with atherosclerosis.

In one embodiment phosphorylated TBK1 expression levels in specific cell-types from the peripheral blood can be used to develop drugs to treat patients with atherosclerosis.

In one embodiment ULK1 expression levels in specific cell-types from the peripheral blood can be used to develop drugs to treat patients with atherosclerosis.

In one embodiment phosphorylated ULK1 expression levels in specific cell-types from the peripheral blood can be used to develop drugs to treat patients with atherosclerosis.

In one embodiment cGAS expression levels in specific cell-types from the peripheral blood can be used to develop drugs to treat patients with atherosclerosis.

In one embodiment NLRP3 expression levels in specific cell-types from the peripheral blood can be used to develop drugs to treat patients with atherosclerosis.

In one embodiment RelA expression levels in specific cell-types from the peripheral blood can be used to develop drugs to treat patients with atherosclerosis.

In one embodiment phosphorylated Rel-A expression levels in specific cell-types from the peripheral blood can be used to develop drugs to treat patients with atherosclerosis.

In one embodiment BDNF expression levels in specific cell-types from the peripheral blood can be used to develop drugs to treat patients with atherosclerosis.

In one embodiment VEGF expression levels in specific cell-types from the peripheral blood can be used to develop drugs to treat patients with atherosclerosis.

In one embodiment MyD88 expression levels in specific cell-types from the peripheral blood can be used to develop drugs to treat patients with atherosclerosis.

In one embodiment Trex1 expression levels in specific cell-types from the peripheral blood can be used to develop drugs to treat patients with atherosclerosis.

In one embodiment AIM2 expression levels in specific cell-types from the peripheral blood can be used to develop drugs to treat patients with atherosclerosis.

In one embodiment Caspase1 expression levels in specific cell-types from the peripheral blood can be used to develop drugs to treat patients with atherosclerosis.

In one embodiment SURF4 expression levels in specific cell-types from the peripheral blood can be used to develop drugs to treat patients with atherosclerosis.

In one embodiment STEEP1 expression levels in specific cell-types from the peripheral blood can be used to develop drugs to treat patients with atherosclerosis.

In one embodiment STIM1 expression levels in specific cell-types from the peripheral blood can be used to develop drugs to treat patients with atherosclerosis.

In one embodiment HER2 expression levels in specific cell-types from the peripheral blood can be used to develop drugs to treat patients with atherosclerosis.

In one embodiment Stat6 expression levels in specific cell-types from the peripheral blood can be used to develop drugs to treat patients with atherosclerosis.

In one embodiment phospho-Stat6 expression levels in specific cell-types from the peripheral blood can be used to develop drugs to treat patients with atherosclerosis.

In one embodiment Src expression levels in specific cell-types from the peripheral blood can be used to develop drugs to treat patients with atherosclerosis.

In one embodiment gene expression is measured in peripheral blood mononuclear cells, including, CD8⁺ T cells, natural killer cells, γ/δ T cells, NKT cells, CD4⁺ T cells, monocytes, or B cells.

In one embodiment the atherosclerotic process involves the coronary arteries.

In one embodiment the atherosclerotic process involves the carotid arteries.

In one embodiment the atherosclerotic process involves arteries in the lower extremities.

In one embodiment the atherosclerotic process involves the aorta.

In one embodiment the atherosclerotic process involves a renal artery.

In one embodiment the atherosclerotic process involves arteries in the upper extremities.

Inhibitors or inducers of one or more components of the STING pathway may be used to treat a subject with atherosclerosis.

In one embodiment an inhibitor of STING is used to treat a patient with atherosclerosis.

In one embodiment an inhibitor of phospho-STING is used to treat a patient with atherosclerosis.

In one embodiment an inhibitor of Akt is used to treat a patient with atherosclerosis.

In one embodiment an inhibitor of phospho-Akt is used to treat a patient with atherosclerosis.

In one embodiment an inhibitor of IRF3 is used to treat a patient with atherosclerosis.

In one embodiment an inhibitor of phospho-IRF3 is used to treat a patient with atherosclerosis.

In one embodiment an inhibitor of TBK1 is used to treat a patient with atherosclerosis.

In one embodiment an inhibitor of phospho-TBK1 is used to treat a patient with atherosclerosis.

In one embodiment an inhibitor of ULK1 is used to treat a patient with atherosclerosis.

In one embodiment an inhibitor of phospho-ULK1 is used to treat a patient with atherosclerosis.

In one embodiment an inhibitor of cGAS is used to treat a patient with atherosclerosis.

In one embodiment an inhibitor of NLRP3 is used to treat a patient with atherosclerosis.

In one embodiment an inhibitor of RelA is used to treat a patient with atherosclerosis.

In one embodiment an inhibitor of phospho-RelA is used to treat a patient with atherosclerosis.

In one embodiment an inhibitor of BDNF is used to treat a patient with atherosclerosis.

In one embodiment an inhibitor of VEGF is used to treat a patient with atherosclerosis.

In one embodiment an inhibitor of MyD88 is used to treat a patient with atherosclerosis.

In one embodiment an inhibitor of Trex1 is used to treat a patient with atherosclerosis.

In one embodiment an inhibitor of AIM2 is used to treat a patient with atherosclerosis.

In one embodiment an inhibitor of Caspase1 is used to treat a patient with atherosclerosis.

In one embodiment an inhibitor of SURF4 is used to treat a patient with atherosclerosis.

In one embodiment an inhibitor of STEEP1 is used to treat a patient with atherosclerosis.

In one embodiment an inhibitor of STIM1 is used to treat a patient with atherosclerosis.

In one embodiment an inhibitor of HER2 is used to treat a patient with atherosclerosis.

In one embodiment an inhibitor of Stat6 is used to treat a patient with atherosclerosis.

In one embodiment an inhibitor of phospho-Stat6 is used to treat a patient with atherosclerosis.

In one embodiment an inhibitor of Src is used to treat a patient with atherosclerosis.

In one embodiment the atherosclerotic process involves the coronary arteries.

In one embodiment the atherosclerotic process involves the carotid arteries.

In one embodiment the atherosclerotic process involves arteries in the lower extremities.

In one embodiment the atherosclerotic process involves renal arteries.

In one embodiment the atherosclerotic process involves the aorta.

In one embodiment the atherosclerotic process involves arteries in the upper extremities.

Inducers and inhibitors of the components of the STING pathway have been developed. For instance, BX795 is a small molecule inhibitor of TBK1 (Clark K, Plater L, Peggie M, Cohen P. Use of the pharmacological inhibitor BX795 to study the regulation and physiology roles of TBK1 and IKB kinase E. J Biol Chem. 284:14136-14146, 2009). The compound was originally described as an inhibitor of 3-phosphoinositide-dependent kinase-1 (Feldman R I, Wu J M, Polokoff M A, Kochanny M J, Dinter H, Zhu D, Biroc S L, Alicke B, Bryant J, Yuan S, Buckman B O, Lentz D, Ferrer M, Whitlow M, Adler M, Finster S, Chang Z, Arnaiz D O. Novel small molecule inhibitors of 3-phosphoinositide-dependent kinase-1. J Biol Chem. 280: 19867-19874, 2005), but was subsequently found to inhibit TBK1 and the related enzyme, IKB kinase E (IKKE). TBK1 and IKKE are both noncanonical IkB kinase homologs that have been implicated in NFKB pathway activation (Bain J, Plater L, Elliott M, Shpiro N, Hastie C J, McLauchlan H, Klevernic I, Arthur J S C, Alessi D R, Cohen P. The selectivity of protein kinase inhibitors: a further update. Biochem J. 408:297-315, 2007). Other TBK1 inhibitors (such as MRT67307, AZ13102909, SR8185, domainex, MP-0485520, amlexanox, compound II, and GSK8612) have been developed and considered for the treatment of patients with cancer, infections, and autoimmune diseases (Xiang S, Song S, Tang H, Smaill J B, Wang A, Xie H, Lu X. TANK-binding kinase 1 (TBK1): an emerging therapeutic target for drug discovery. Drug Discovery Today. 26:2445-2455, 2021; Hasan M, Yan N. Therapeutic potential of targeting TBK1 in autoimmune diseases and interferonopathies. Pharmacol Res. 111:336-342, 2016; Thomson D W, Poeckel D, Zinn N, Rau C, Strohmer K, Wagner A J, Graves A P, Perrin J, Bantscheff M Duempelfeld B, Kasparcova V, Ramanjulu J M, Pesiridis G S, Muelbaier M, Bergamini G. Discovery of GSK8612, a highly selective and potent TBK1 inhibitor. ACS Medicinal Chem Letters. 10:780-785, 2019).

Similarly, inhibitors and inducers of STING have been developed for the potential of therapeutic use. A diamidobenzimidazole, diABZI-4, activates STING and has been found to ameliorate infection with SARS-CoV2 in murine models (Humphries F, Shmuel-Galia L, Jiang Z, Wilson R, Landis P, Ng S, Parsi K M, Maehr R, Cruz J, Morales A, Ramanjulu J M, Bertin J, Pesiridis G S, Fitzgerald K A. A diamidobenzimidazole STING agonist protects against SARS-CoV-2 infection. Science Immunol. 6, eabi9002, 2021; Li M, Ferretti M, Ying B, Descamps H, Lee E, Dittmar M, Lee J, Whig K, Kamalia B, Dohnalova L, Uhr G, Zarkoob H, Chen Y, Ramage H, Ferrer M, Lynch K, Schultz D C, Thaiss C A, Diamong M S, Cherry S. Pharmacological activation of STING blocks SARS-CV-2 infection. Science Immunol. 6, eabi9007, 2021; Feng X, Liu D, Li Z, Bian J. Bioactive modulators targeting STING adaptor in cGAS-STING pathway. Drug Discovery Today. 25:230-237, 2020). STING inhibitors have also been studied in order to treat mice with autoinflammatory disease caused by Trex1 knockout (Haag S M, Gulen M F, Reymond L, Gibelin A, Abrami L, Decout A, Heymann M, van der Goot F G, Turcatti G, Behrendt R, Ablasser A. Targeting STING with covalent small-molecule inhibitors. Nature. 559:269-273, 2018). Inhibitors of cGAS-STING have been proffered as anti-inflammatory agents (Sheridan C. Drug developers switch gears to inhibit STING. Nature Biotechnol. 37:199-208, 2019).

Inducers and inhibitors of various components of the STING pathway have been developed for the treatment of cancer, infection, and inflammation including Parkinson's disease, nonalcoholic steatohepatitis, systemic lupus erythematosus, lupus nephritis, and COVID-19; however, these compounds have not been previously proposed for the treatment of patients with atherosclerosis most likely because the involvement of the STING pathway in this disorder has not been previously disclosed.

In one embodiment the invention is directed to a composition comprising one or more peripheral blood cells in which individual cells are co-stained for a cell-lineage marker for the peripheral blood cell and for a marker for one or more components of the STING pathway.

In one embodiment the composition is prepared by tyramide signal amplification. The methods of preparation produce peripheral blood cells that are suitable for detection by flow cytometry.

In one embodiment of the composition, the peripheral blood cell is a mononuclear cell.

In one embodiment of the composition, the mononuclear cell is selected from the group consisting of monocytes, B cells, and T cells.

In one embodiment of the composition, the mononuclear cell is a monocyte.

In one embodiment of the composition, the mononuclear cell is a B cell.

In one embodiment of the composition, the mononuclear cell is a T cell.

In one embodiment of the composition, the T cell is a CD8⁺ T cell or CD4⁺ T cell.

In one embodiment of the composition, the cell-lineage marker for the peripheral blood cell is a marker of peripheral blood mononuclear cells.

In one embodiment of the composition, the cell-lineage marker is for B cells.

In one embodiment of the composition, the cell-lineage marker is for T cells.

In one embodiment of the composition, the cell-lineage marker is for CD8⁺ T cells or CD4⁺ T cells.

In one embodiment of the composition, the cell-lineage marker is for monocytes.

In one embodiment of the composition, the marker for a component of the STING pathway is selected from the group consisting of STING, phospho-STING, Akt, phospho-Akt, IRF3, phospho-IRF3, TBK1, phospho-TBK1, ULK1, phospho-ULK1, cGAS, NLRP3, RelA, phospho-RelA, BDNF, VEGF, MyD88, Trex1, AIM2, Caspase1, SURF4, STEEP1, STIM1, HER2, Stat6 and Src.

In one embodiment of the composition, the co-stain comprises an antibody.

In one embodiment of the composition, the stain for the cell-lineage marker and the stain for the STING component are both antibodies.

Antibodies for B cells include CD19 and CD20. Either of these markers can be used to identify B cells. The antibodies for CD4⁺ T cells can be CD3 and/or CD4. The antibodies for CD8⁺ T cells can be CD3 and CD8. The antibodies for natural killer (NK) cells can be CD56 and/or CD16. The antibodies for monocytes can be CD14 and/or CD4.

The invention is also directed to a kit for detecting the gene expression of one or more components of the STING pathway in a biological sample. The biological sample may be derived from a subject having, or at risk of having, atherosclerosis or in the event that one desires to detect the effect of a component on the STING pathway, per se, the cells that are being analyzed that express the STING pathway may be derived from a normal subject, i.e., a subject not having atherosclerosis or even at risk for developing atherosclerosis. The kit includes one or more cell-lineage markers: for B cells, CD19 or CD20; for CD4⁺ T cells, CD3 and/or CD4; for CD8⁺ T cells, CD3 and CD8; for natural killer cells, CD56 and/or CD16; and for monocytes, CD14 and/or CD4.

The kit components include the following:

Direct Staining:
Antibody to lineage marker(s) conjugated with fluorochrome
Antibody to STING pathway component(s) conjugated with fluorochrome
Cell fixation reagent: e.g., paraformaldehyde
Cell Permeabilization reagent: e.g., saponin (a detergent); an alcohol can also be used (e.g., methanol)

Indirect Staining without Amplification:
Antibody to lineage marker(s) conjugated with fluorochrome
Antibody to STING pathway component(s) conjugated with fluorochrome
Cell fixation reagent: e.g., paraformaldehyde
Cell Permeabilization reagent: e.g., saponin (a detergent); an alcohol can also be used (e.g., methanol)
Secondary antibody conjugated with fluorochrome

Indirect Staining With Amplification:

The kit contains components that produce stained peripheral blood cells that are suitable for detection by flow cytometry.
Antibody to lineage marker(s)
Antibody to STING pathway component(s)
Cell fixation reagent: e.g., paraformaldehyde
Cell Permeabilization reagent: e.g., saponin (a detergent); an alcohol can also be used (e.g., methanol)
A compound that binds to the primary antibody and that is conjugated with peroxidase, and, in a specific embodiment, secondary antibody is conjugated with horseradish peroxidase
Reporter: tyramide conjugated to a fluorochrome, e.g., fluorescein
Amplification medium
Wash medium

In one embodiment of the staining procedure, a secondary antibody may be used. Thus, the peroxidase is associated with the secondary antibody rather than the primary antibody.

For Cell Surface Staining:
The amplification medium includes ficoll/hypaque and 4-aminobenzoic acid hydrazine.

For Intracellular Staining
The amplification medium includes a glycine-glycine buffer.
Also, the final wash for intracellular staining includes urea.
For ficoll/hypaque, a specific concentration is ≤90%, but an effective concentration is ≤50%. For 4-aminobenzoic acid hydrazine, a specific concentration is 50 mM. However, an effective concentration is ≥10 mM. For diglycine medium, a specific concentration is 4 mg/ml (pH 8.0). However, an effective concentration range is from 0.4 to 40 mg/ml (pH range 7.5-9.0). For the urea, a specific concentration is 6.0 M. However, an effective range is 4-7 M.

The amplification medium and the wash medium are disclosed in U.S. Pat. No. 6,828,109.

Various techniques for assessing the level of gene expression include, but are not limited to, flow cytometry, flow cytometry with tyramide deposition technology, single cell mass cytometry, immunohistochemistry, Western analysis, immunohistochemistry, enzyme-linked immunosorbent assays (ELISA), and nucleic acid analysis including single cell polymerase chain reaction (PCR).

Methods for detecting gene expression may be performed on individual cells or on preparations that are not resolved at the level of individual cells. That is, detection of one or more components of the STING pathway may be of signal on individual cells. Such individual cells would be co-stained for a cell lineage marker and for one or more components of the STING pathway. In that case, the preparation need not be homogenous for any specific cell type. In that case, the specific cell types need not be isolated or purified so a sample of peripheral blood or peripheral blood mononuclear cells is analyzed. Alternatively, there may be detection methods in which individual cells cannot be analyzed. In such a case, the specific cell type may be isolated, enriched or purified for analysis, such as, a homogeneous population of a desired peripheral blood cell type (e.g., monocytes).

For methods of detection in which expression in individual cells is not done, one may isolate a desired cell type (for example, monocyte, B cell, etc.) for analysis of gene expression of one or more components of the STING pathway. In such a case, the preparation may be enriched for the cell type, including, substantially homogeneous preparations.

In one embodiment, gene expression of the lineage marker and/or the component of the STING pathway is amplified.

In one embodiment, staining is direct as by detectable antibody to the marker.

In one embodiment, staining is indirect but without amplification; for example, a secondary detectable signal that is associated (e.g., bound/linked) with the antibody.

In one embodiment, staining is indirect and the signal is amplified, as with an antibody-associated enzyme that comprises one or more detectable reporters.

In one embodiment, the sample is analyzed by flow cytometry. In this case, the cells may be stained for a lineage marker and one analyte; however, cells may also be stained for a lineage marker and more than one analyte at a time, e.g., a monocyte stained for more than one component of the STING pathway.

In one embodiment, the levels of gene expression are detected by flow cytometry with tyramide deposition technology, disclosed, for example, in U.S. Pat. Nos. 6,280,961, 6,335,173, and 6,828,109, incorporated by reference for the amplification methods disclosed.

With respect to the present invention, this technology is used to produce a labeled peripheral blood cell as described in this application that is suitable for detection by flow cytometry. Such a peripheral blood cell, as described herein, has the analyte signal amplified using the disclosed amplification medium with the tyramide deposition. Furthermore, the wash medium disclosed in this application is used on the peripheral blood cells just prior to their being introduced into the flow cytometer. Thus, the peripheral blood cells that are washed in the wash medium have undergone the entire staining and amplification process; namely, antibody binding, peroxidase associated with the antibody, and tyramide deposition catalyzed by the peroxidase.

Accordingly, the invention is also directed to the co-stain of peripheral blood cells, including peripheral blood cells in which the signal has been amplified, in a flow cytometer that allows detection of the signal of the cell-lineage marker and the one or more components of the STING pathway on individual peripheral blood cells.

Tyramide signal amplification (TSA), sometimes called Catalyzed Reporter Deposition (CARD), is a highly sensitive method enabling the detection of low-abundance targets.

TSA involves peroxidase-catalyzed deposition of tyramide on and near a target protein or nucleic acid sequence in situ. In the presence of low concentrations of H2O2, peroxidase is able to convert a labeled tyramide substrate into a highly reactive form that can covalently bind to tyrosine residues on proteins at or near the peroxidase. This generates high density tyramide labeling. Tyramide can be labeled with a fluorophore or a hapten. Multiple rounds of tyramide signal amplification can be performed.

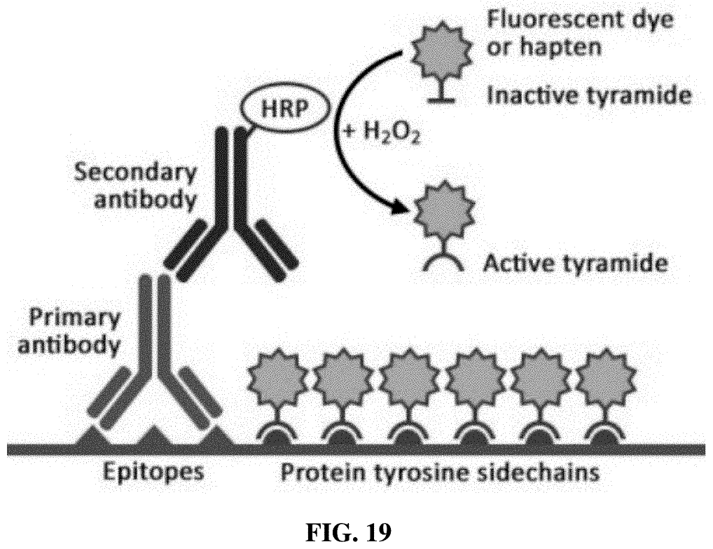

FIG. 19 shows an illustration of the tyramide signal amplification system. A cell or tissue sample is labeled with primary and (optionally) secondary antibody using conventional methods. The horseradish peroxidase, conjugated to the secondary antibody, catalyzes the conversion of labeled tyramide into a reactive radical. The tyramide radical then covalently binds to nearby tyrosine residues, providing high-density labeling.

Gene expression can be detected by directly assaying protein or RNA (or modifications). This could be done through techniques available in the art, such as by flow cytometry and other antibody-based detection methods, and PCR and other hybridization-based methods.

In one embodiment protein expression is detected. Protein expression that is assayed can be intracellular, extracellular (i.e. surface), or both.

In one embodiment gene expression is detected via expression of RNA. RNA can be any RNA, including, messenger RNA and smaller RNA molecules, such as microRNAs.

In a further embodiment, post-translational modifications may be detected, including phosphorylation, acetylation, nitrosylation, ubiquitination, sulfation, glycosylation, myristoylation, palmistoylation, isoprenylation, farnesylation, geranylgeranylation, alkylation, amidation, acylation, oxidation, SUMOylation, Pupylation, Neddylation, biotinylation, pegylation, succinylation, selenoylation, citrullination, deamidation, ADP-ribosylation, iodination, hydroxylation, gamma-carboxylation, carbamylation, S-nitrosylation, S-glutathionylation, and malonylation, as well as any other post-translational modification.

In one embodiment gene expression is detected by flow cytometry. Another embodiment involves the detection of molecular expression levels in enriched cells by western blotting. Another embodiment involves the detection of molecular expression levels via reverse phase protein arrays involving purified cells. Kornblau S et al., Blood 2009, 113:154-164 Immunoassays on lysates of purified or enriched cells is another embodiment. Gene expression can also be assessed by measuring mRNA with enough precision to obtain correlations with r>0.6. mRNA determinations can be obtained with real-time PCR.

In another embodiment gene expression is detected in individual cells.

In another embodiment gene expression is detected in at least 50 cells.

### Example

### Initial Study

The inventor considered the possibility that molecular expression levels in the specific subsets of immune cells among peripheral blood mononuclear cells, which include B lymphocytes, CD4⁺ T cells, CD8⁺ T lymphocytes, and monocytes, may be useful in modeling atherosclerosis initiation and progression. Consequently, the inventor evaluated the expression levels of several molecules in each of the different types of peripheral blood mononuclear cells from patients at cardiac evaluation (n=74). Among these subjects 40 had coronary artery calcium scores of zero which indicates little or no atherosclerosis, 15 patients had coronary artery calcium scores greater than 5 but less than 200 which indicates mild, subclinical atherosclerosis, and 19 patients had coronary artery calcium scores greater than 200 which indicates moderate subclinical atherosclerosis.

The inventor selected analytes to evaluate what he hypothesized may be important in the pathogenesis of atherosclerosis. The analytes assessed were phospho-Akt(thr308) (pAkt or phospho-Akt), vitamin D receptor (VDR), brain derived nerve factor (BDNF), arylhydrocarbon receptor (AHR), translocator protein (TSPO), RelA (p65 of the NFkB pathway), interferon regulatory factor 3 (IRF3), BclxL (an anti-apoptotic molecule), NLRP3 (NACHT, LRR and PYD domains-containing protein 3), vascular endothelial growth factor (VEGF), stimulator of interferon genes (STING), phospho-STING at serine 366 (pSTING or phospho-STING), high mobility group box 1 (HMGB1), survivin, and catechol-o-methyltransferase (COMT).

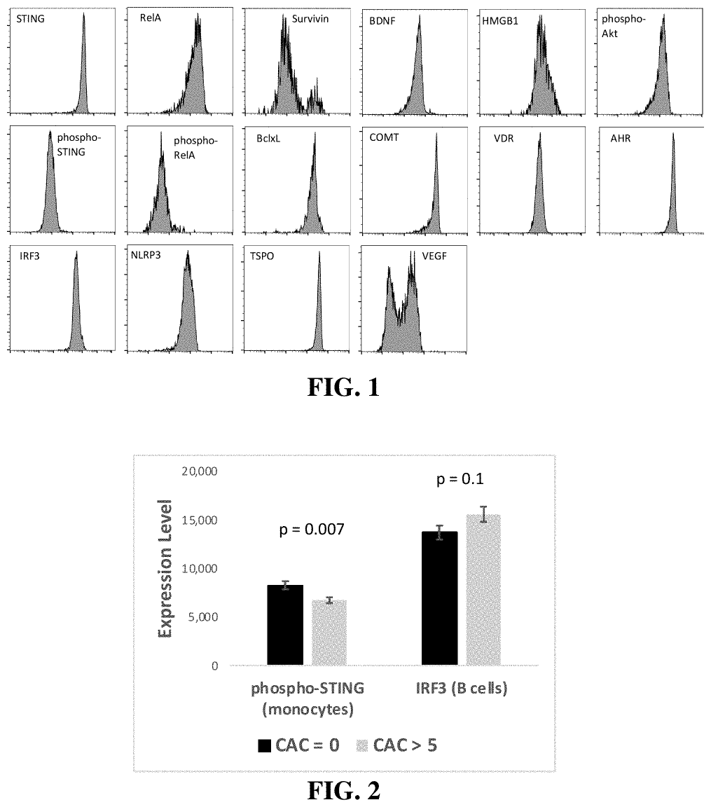

FIG. 1 shows analytes that were assessed by flow cytometry using the CellPrint™ signal amplification technology (15-31) in monocytes which were identified by intermediate expression levels of CD4. The various cell types were identified by co-staining with antibodies specific for CD19 (B cells) and CD4 (CD4⁺ T cells and monocytes). The co-stain for the expression of the cell lineage markers was not amplified. All the analytes assessed are intracellular molecules; consequently, the cells were fixed and permeabilized prior to staining. The expression levels of the analytes were enhanced with CellPrint™.

The samples from patients obtained at evaluation of coronary artery atherosclerosis by assessing coronary artery calcification were de-identified and the clinical status associated with the samples was not revealed to the laboratory technicians. After the dataset including the cell type-specific molecular expression levels was finalized, the inventor was given the key to the clinical status associated with each sample.

The staining of the cells provided relatively sharp peaks with over a thousand gated cells/peak which provide exceptionally accurate quantification of relative expression levels.

Sharp peaks allow for more confident interpretation of the data.

Comparing the expression levels of the various analytes in the 3 cellular subtypes, the inventor found a single statistically significant expression level change between samples from patients with no calcification (coronary artery calcium (CAC) score=0) and patients with significantly calcified atherosclerotic lesions as indicated by CAC>5.

The expression level of phospho-STING in monocytes was significantly decreased (p=0.007) in samples from patients with calcified atherosclerosis compared to samples from patients without calcified atherosclerosis (FIG. 2 right). The phosphorylation of STING at serine 366 has been shown to be inactivating for the molecule (20) although other investigators have evidence that this same phosphorylation is activating (Tanaka Y, Chen Z J. STING specifies IRF3 phosphorylation by TBK1 in the cytosolic DNA signaling pathway. Science Signaling. 5(214):ra20, 2012.; Liu S, Cai X, Wu J, Cong Q, Chen X, Li T, du F, Ren J, Wu Y, Grishin N V, Chen Z J. Phosphorylation of innate immune adaptor proteins MAVS, STING, and TRIF induces IRF3 activation. Science. 347(6227): aaa2630. doi: 10.1126/science.aaa2630, 2015.). Consequently, the decrease in phospho-STING may indicate greater STING activity in samples from the patients with calcification. The expression level of IRF3 in B cells is also shown although the increase for patients with calcification is not significant. No other expression levels showed significant differences or even differences close to significance.

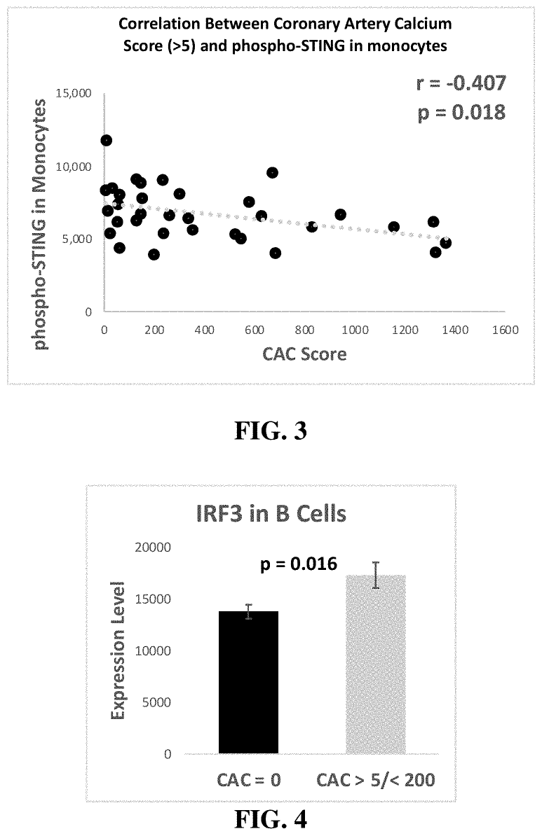

Also, the expression level of phospho-STING was significantly correlated with CAC among the samples from patients with calcified atherosclerosis (CAC>5). FIG. 3 (right) shows an inverse relationship with phospho-STING levels in monocytes and CAC. The decreasing phospho-STING levels in monocytes with increasing severity of calcified atherosclerosis suggests that the difference in expression levels of phospho-STING in monocytes is related to the disease process.

The process of atherosclerotic calcification can be divided into initiation and progression phases by the value of CAC. Patients at evaluation with CAC scores greater than 5 and less than 200 have mild disease that is considered to be subclinical. Patients at evaluation with CAC scores greater than 200 have moderate disease although it is still considered to be subclinical. Consequently, the inventor looked for differences in cell-specific molecular expression levels that corresponded to these clinical categories.

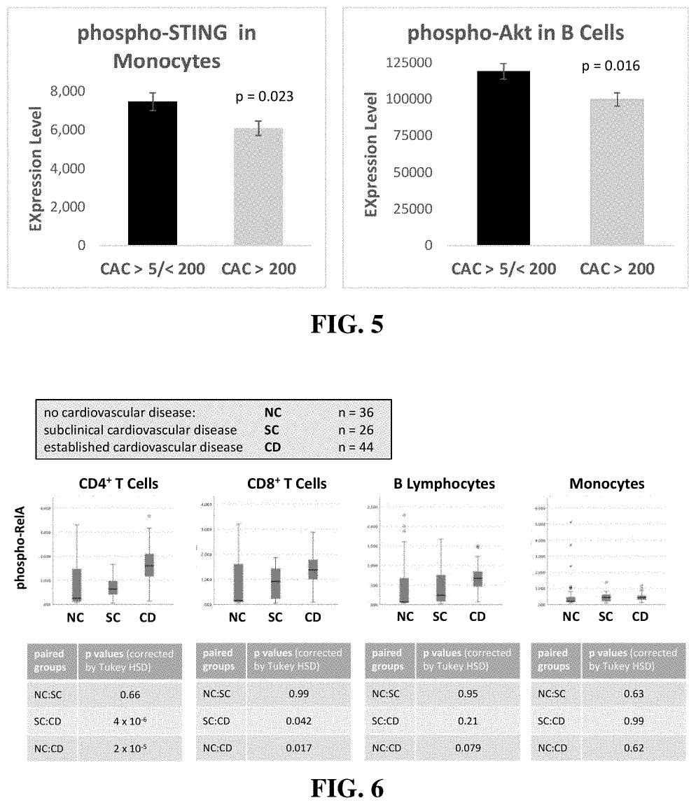

Differences in expression levels in specific cell types from the peripheral blood were considered in regards to the progression of coronary artery atherosclerotic calcification from mild or moderate subclinical disease to advanced subclinical disease. Differences in expression levels between patients with mild subclinical coronary artery calcification (CAC>5/<200) and patients with moderate subclinical disease (CAC>200) were found for two analytes in various cell types. Phospho-STING^{ser366} in monocytes and phospho-Akt^{thr308} in B cells from patients with advanced subclinical disease were significantly decreased compared to patients with moderate subclinical disease (FIG. 5).

These results indicate that the progression of coronary artery calcification can be modeled by molecular expression level changes in specific subsets of cells from the peripheral blood.

Thus, CellPrint™ analysis of molecular expression levels associated with specific cellular types in the peripheral blood was able to model coronary artery calcified atherosclerosis involving CAC. Since coronary artery calcified atherosclerosis is a localized inflammatory process, these findings indicate the power of assessing localized immune/inflammatory processes by analysis of circulating blood cells.

### Follow-On Study

A double-blind confirmatory analysis of the capability of the CellPrint™ platform to reveal molecular disease signatures in blood immune cells of patients with coronary artery disease was performed. Peripheral blood mononuclear cells from 40 patients with no coronary artery disease, 27 patients with mild/moderate/subclinical coronary artery disease, and 45 patients with established coronary artery disease were assessed for expression levels in four mononuclear cell-types: CD4⁺ T cells, CD8⁺ T cells, B cells, and monocytes. Eleven analytes were assessed in the study: STING, phospho-STING, IRF3, phospho-Akt, BDNF, phospho-RelA, NLRP3, Traf6, phospho-TBK1, phospho-ULK1, and MyD88. The signals from the analytes were amplified by the CellPrint™ technology. The co-stains for the mononuclear cell lineage were not amplified. The cells were assessed by flow cytometric analysis.

FIG. 6 shows the results for cell-type specific phospho-RelA expression. Phospho-RelA indicates the level of activation of the major inflammatory pathway, NFkB. Comparisons are made between expression levels of patients with no coronary artery disease (NC), patients with subclinical coronary artery disease (SC; defined as positive coronary artery calcification, and patients with established coronary artery disease (CD; defined clinically and by coronary artery evaluation). The comparisons were made by ANOVA and the p values shown have been corrected by Tukey HSD.

Both T cell subsets, but not monocytes or B lymphocytes, showed increases in phospho-RelA expression with disease progression (SC compared to CD).

FIG. 7 shows the results for cell-type specific IRF3 expression. IRF3 is a transcription factor leading to the production of type I interferon. The comparisons were made by ANOVA and the p values shown have been corrected by Tukey HSD.

IRF3 is decreased in monocytes with disease progression (SC compared to CD). Increased IRF3 in both T cell subsets characterizes disease (NC compared to CD).

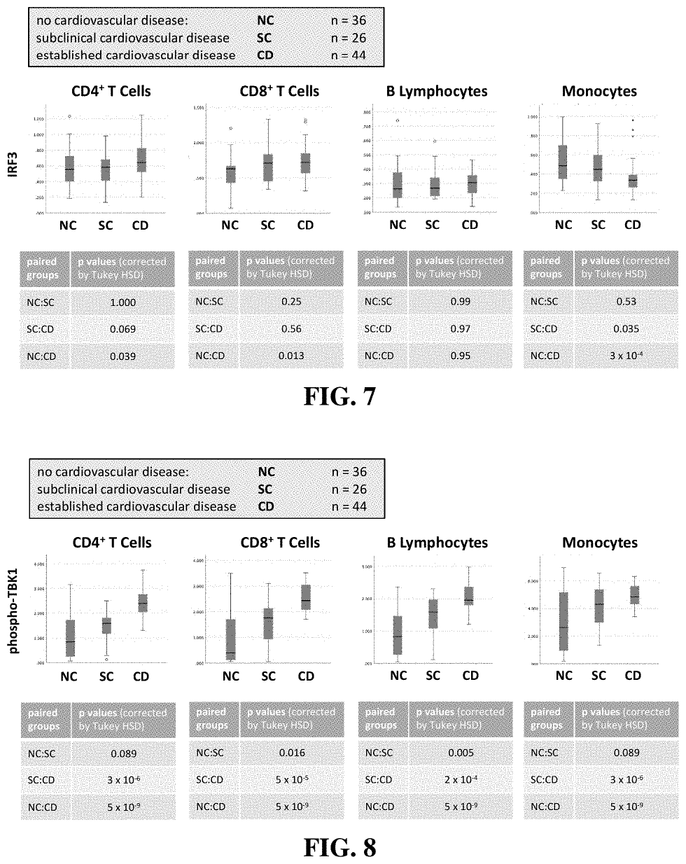

FIG. 8 shows the results for cell-type specific phospho-TBK1. The comparisons were made by ANOVA and the p values shown have been corrected by Tukey HSD.

The initiation (NC compared to SC) and progression (SC compared to CD) of coronary artery disease is characterized by an increase in phospho-TBK1 expression in all cell-types except for initiation (NC compared to SC) in CD4⁺ T cells and monocytes.

FIG. 9 shows the results for cell-type specific phospho-Akt expression. The comparisons were made by ANOVA and the p values shown have been corrected by Tukey HSD.

B cells showed decreased phospho-Akt with both disease initiation (NC compared to SC) and progression (SC compared to CD). Monocytes showed decreased phospho-Akt expression with disease progression (SC compared to CD).

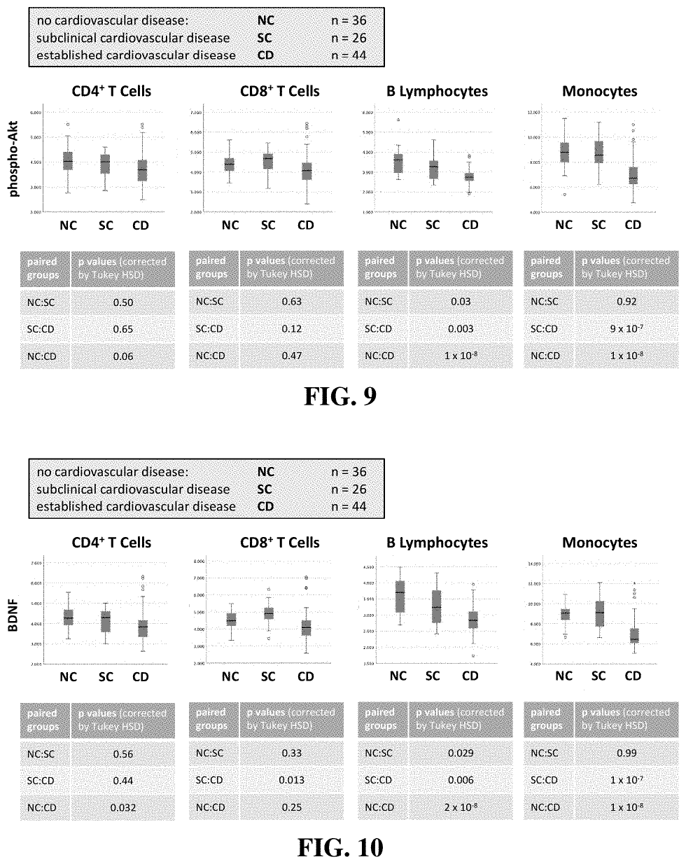

FIG. 10 shows the results for cell-type specific BDNF expression. The comparisons were made by ANOVA and the p values shown have been corrected by Tukey HSD.

B cells showed decreased BDNF expression with both disease initiation and disease progression. Monocytes and CD8⁺ T cells showed decreased BDNF expression with disease progression.

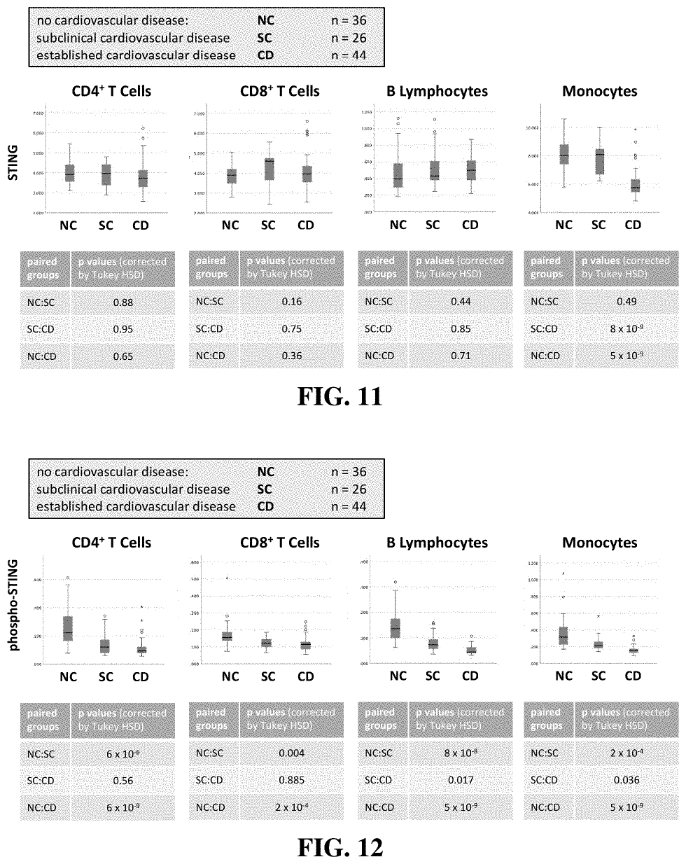

FIG. 11 shows the results for cell-type specific STING expression. The comparisons were made by ANOVA and the p values shown have been corrected by Tukey HSD.

STING in monocytes only is markedly decreased upon progression of coronary artery disease from subclinical to established disease (SC compared to CD).

FIG. 12 shows the results for cell-type specific phospho-STING expression. The comparisons were made by ANOVA and the p values shown have been corrected by Tukey HSD.

Phospho-STING is markedly decreased with the initiation of coronary artery disease (NC compared to SC) for all cell-types and with the progression of coronary artery disease (SC compared to CD) for B cells and monocytes.

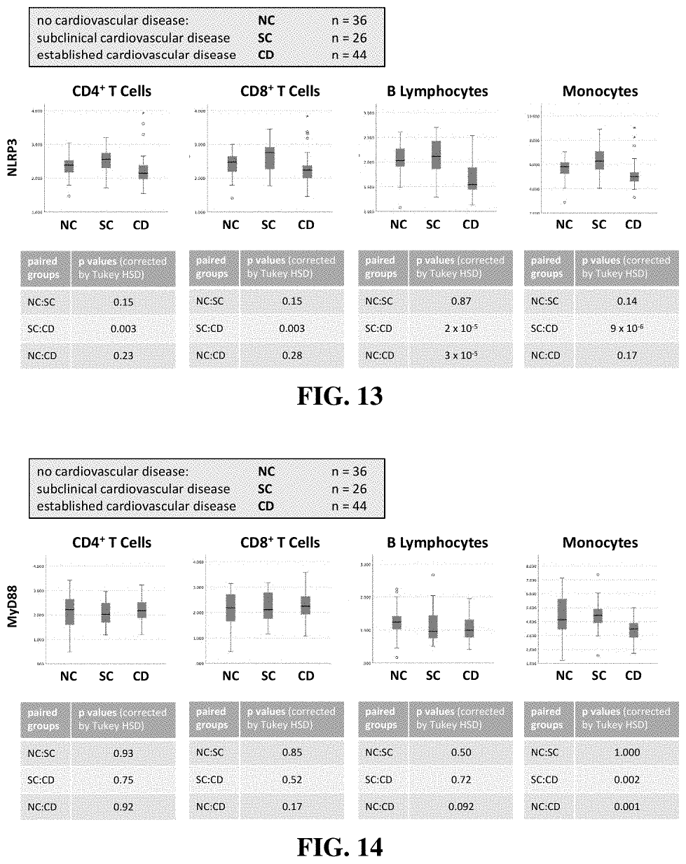

FIG. 13 shows the results for cell-type specific NLRP3 expression. The comparisons were made by ANOVA and the p values shown have been corrected by Tukey HSD.

NLRP3 shows a marked decrease in expression upon progression of subclinical coronary artery disease to established coronary artery disease (SC compared to CD) for all cell-types.

FIG. 14 shows the results for cell-type specific MyD88 expression stratified by clinical categories of coronary artery disease. The comparisons were made by ANOVA and the p values shown have been corrected by Tukey HSD.

MyD88 is decreased in monocytes only upon coronary artery disease progression (SC compared to CD).

The differences in expression levels stratified to specific mononuclear cell-types found in the peripheral blood can be used to distinguish patients (diagnostic), to manage patients (diagnostic), to develop therapeutic agents (biomarker), and to identify new targets for therapeutic intervention (drug development).

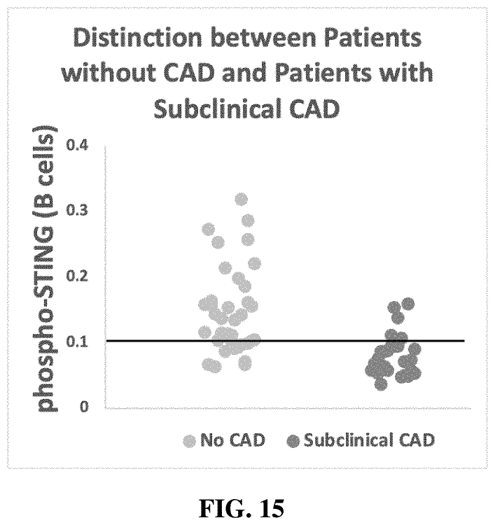

For instance, FIG. 15 shows how the expression level of phospho-STING in B lymphocytes can be used to distinguish patients with no coronary artery disease to those patients with coronary artery disease.

A logistic regression model with B cell phospho-STING only has a p value<0.001 and 22 true positive, 7 false positives, 29 true negatives, and 5 false negatives. Consequently, the positive predictive value of B cell phospho-STING for distinguishing patients without coronary artery disease from those with subclinical coronary artery disease is 76%. The negative predictive value is 85%. The sensitivity is 81%, and the specificity is 81%.

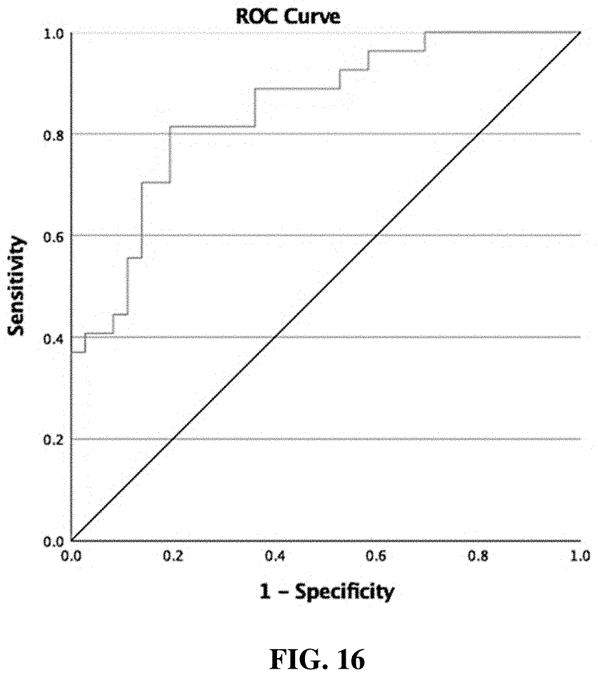

A receiver operator characteristics (ROC) analysis of B cell phospho-STING stratifying patients with subclinical coronary artery disease from those with none is shown in FIG. 16.

The area under the curve (AUC) is 0.85 with a 95% confidence interval of 0.75-0.94 and a p value<0.001.

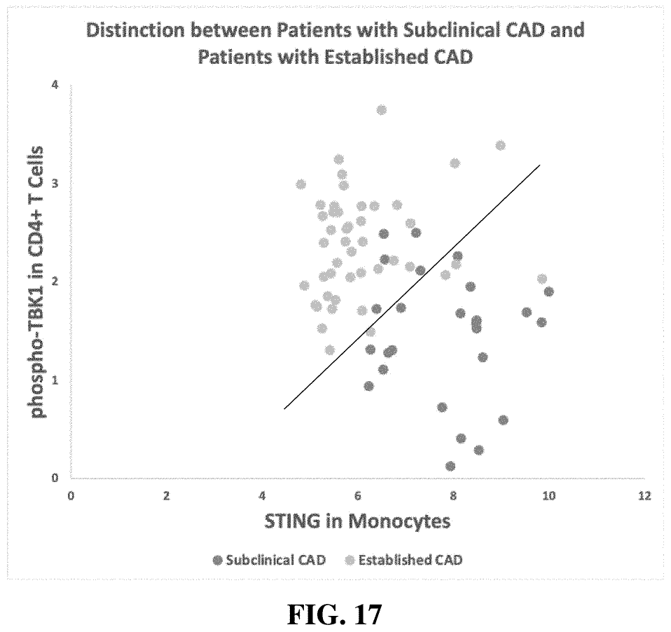

Cell-type specific expression levels can also be used to distinguish patients with subclinical coronary artery disease from patients with established coronary artery disease. For instance, STING in monocytes and phospho-TBK1 in CD4⁺ T cells distinguish these patients by logistic regression, as shown in FIG. 17.

The p value for the model is <0.001, and the p values for monocyte STING and CD4⁺ T cell phospho-TBK1 are 0.001 and <0.001 respectively. This model gives 41 true positives, 5 false positives, 22 true negatives, and 4 false negatives for an 89% positive predictive value, an 85% negative predictive value, a sensitivity of 91% and a specificity of 81%. Including 3 factors in the model gives a positive predictive value of 95%, a negative predictive value of 89%, a sensitivity of 93%, and a specificity of 93%. The model p value remains <0.001, and p values for all the independent factors in the model are 0.003 (monocyte STING), 0.001 (phospho-RelA in CD4⁺ T cells), and 0.022 (BDNF in monocytes).

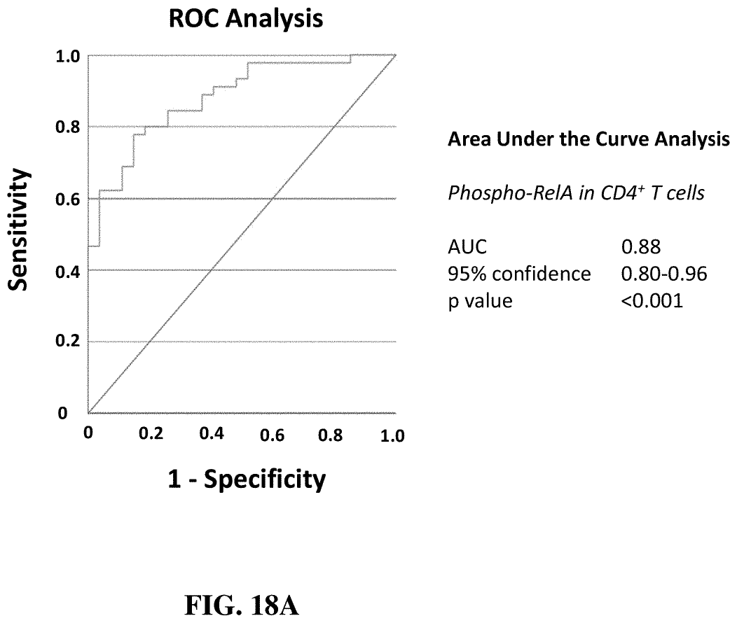

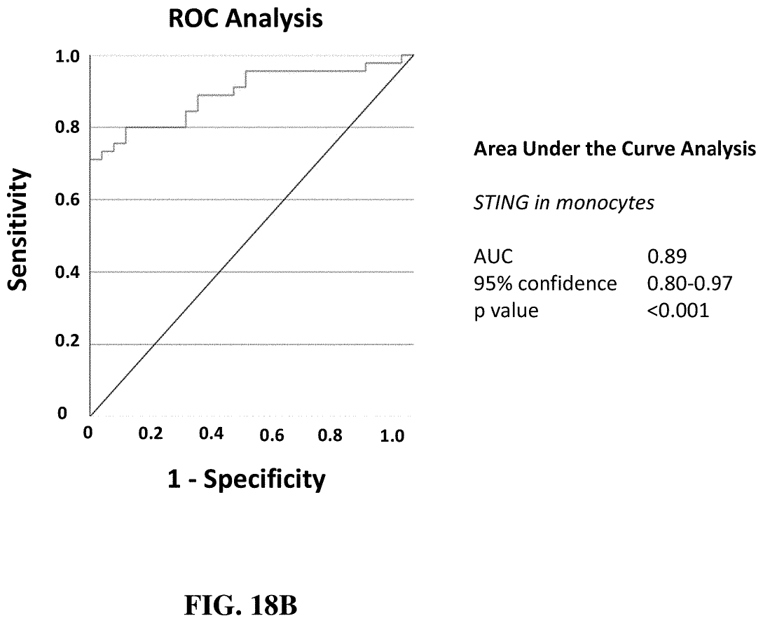

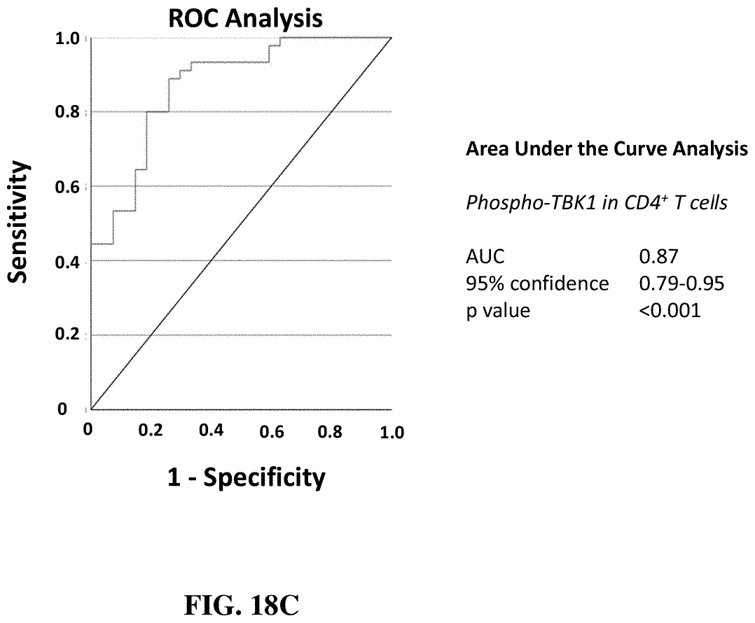

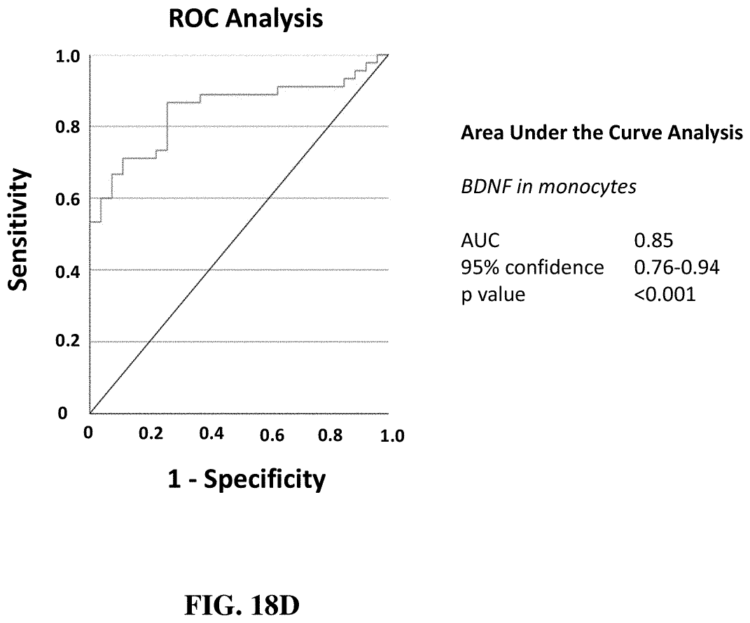

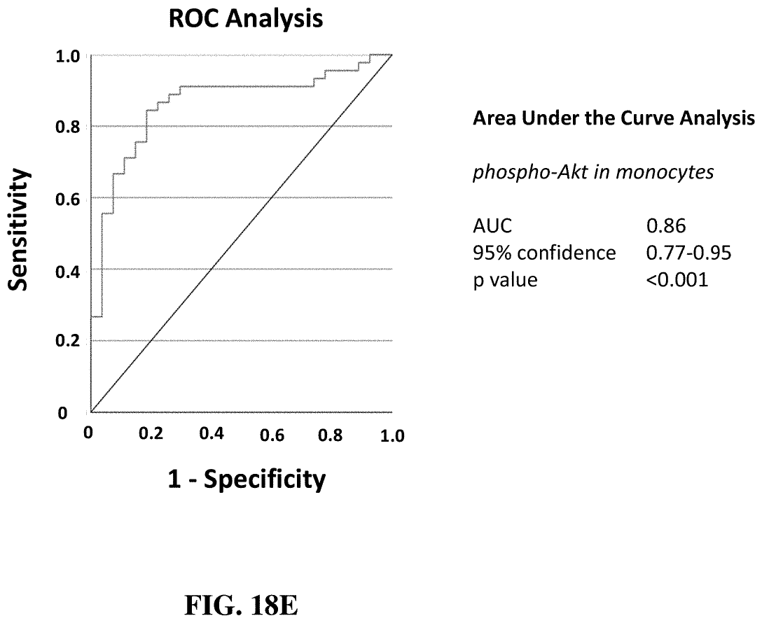

FIGS. 18A through 18E show that by ROC analysis individual factors demonstrate efficacy in distinguishing patients with subclinical coronary artery from patients with established coronary artery disease. FIG. 18A shows a ROC (receiver operating characteristic) analysis for phospho-RelA expression levels in CD4⁺ T cells that distinguishes patients with subclinical CAD from patients with clinical CAD. FIG. 18B shows a ROC analysis for STING expression levels in monocytes that distinguishes patients with subclinical CAD from patients with clinical CAD. FIG. 18C shows a ROC analysis for phospho-TBK1 expression levels in CD4⁺ T cells that distinguishes patients with subclinical CAD from patients with clinical CAD. FIG. 18D shows a ROC analysis for BDNF expression levels in monocytes that distinguishes patients with subclinical CAD from patients with clinical CAD. FIG. 18E shows a ROC analysis for phospho-Akt expression levels in monocytes that distinguishes patients with subclinical CAD from patients with clinical CAD.

TABLE 1

| | | | | 95% confidence |
|---|---|---|---|---|
| Analyte | Cell-Type | AUC | p value | interval |
| phospho-TBK1 | B cell | 0.88 | <0.001 | 0.79-0.96 |
| phospho-TBK1 | CD8⁺ T cell | 0.89 | <0.001 | 0.80-0.98 |
| phospho-TBK1 | CD4⁺ T cell | 0.86 | <0.001 | 0.77-0.95 |
| phospho-Akt | B cell | 0.86 | <0.001 | 0.78-0.94 |
| phospho-Akt | monocyte | 0.86 | <0.001 | 0.77-0.95 |
| BDNF | B cell | 0.86 | <0.001 | 0.76-0.93 |
| BDNF | monocyte | 0.87 | <0.001 | 0.78-0.96 |
| STING | monocyte | 0.91 | <0.001 | 0.84-0.98 |
| phospho-STING | CD4⁺ T cell | 0.89 | <0.001 | 0.81-0.96 |
| phospho-STING | B cell | 0.97 | <0.001 | 0.95-1.00 |
| phospho-STING | monocyte | 0.94 | <0.001 | 0.89-0.98 |

Table 1 above shows how several individual cell-type specific molecular levels stratified patients with no coronary artery disease from patients with established coronary artery disease by ROC analysis with high AUC values.

The findings of the follow-on study corroborate the major finding of the initial investigation. The decrease in phospho-STING levels in monocytes with increasing levels of coronary artery calcium suggested involvement of the STING pathway in the pathogenesis of coronary artery disease. Since phosphorylation of STING on the serine residue at position 366 has been shown to be inhibitory in some circumstances (20), a loss of phospho-STING suggests increased STING pathway activity. In the second study this finding was confirmed and extended with a distinct set of samples. The depression of monocyte phospho-STING(ser366) with the initiation of coronary artery calcification was replicated but also the depression of phospho-STING(ser366) was seen in the second study to include the other mononuclear cell-types. Additionally, further depression of phospho-STING in both B cells and monocytes were observed in the patients with established coronary artery disease.

The second study included many more samples than the initial study. Consequently, the relationship of other components of the STING pathway were seen to correlate with clinical parameters more clearly in the second study. The second study included samples from patients with established coronary artery disease so the effects on pathogenesis could extend beyond the initiation phase of the process and include disease progression. The second study included more analytes involved in the STING pathway thereby demonstrating more fully the perturbation of the pathway with coronary artery disease.

### REFERENCES

1) Chen H-J, Tas S W, de Winther M P J, “Type-I Interferons in Atherosclerosis,” J. Exp. Med. 2020 Jan. 6; 217(1): e20190459. doi: 10.1084/jem.20190459.
2) Khera A, Greenland P., “Coronary Artery Calcium: If Measuring Once is Good, Is Twice Better,” Circulation 2018; 137(7):680-683.
3) Goossens P, Gijbels M J J, Zernecke A, Eijgelaar W, Vergouwe M N, Made I V D, Vanderlocht J, Beckers L, Buurman W A, Daemen M J A P, Kalinke U, Weber C, Lutgens E, Winther M P J D, “Myeloid Type I Interferon Signaling Promotes Atherosclerosis by Stimulating Macrophage Recruitment to Lesions,” Cell Metab. 2010 Aug. 4; 12(2):142-153.
4) Stewart C R, Stuart L M, Wilkinson K, Gils J M V, Deng J, Halle A, Rayner K J, Boyer L, Zhong R, Frazier W A, Hulbert A L, Khoury J E, Golenbock D T, Moore K J, “CD36 Ligands Promote Sterile Inflammation Through Assembly of a Toll-like Receptor 4 and 6 Heterodimer,” Nat. Immunol. 2010 February; 11(2):155-61, doi: 10.1038/ni.1836.
5) Hansson G K, Holm J, Jonasson L., “Detection of Activated T Lymphocytes in the Human Atherosclerotic Plaque,” Am J Pathol. 1989 July; 135(1):169-175.
6) Mallat Z, Heymes C, Ohan J, Faggin E, Leseche G, Tedgui A, “Expression of Interleukin-10 in Advanced Human Atherosclerotic Plaques: Relation to Inducible Nitric Oxide Synthase Expression and Cell Death,” Arterioscler Thromb Vasc Biol. 1999 March; 19(3):611-616, doi: 10.1161/01.ATV.19.3.611.
7) Berbverian P A, Myers W, Tytell M, Challa V, Bond M G, “Immunohistochemical Localization of Heat Show Protein-70 in Normal-Appearing and Atherosclerotic Specimens of Human Arteries,” Am J Pathol. 1990 January; 136(1):71-80.
8) Matsumoto M K, Ishikawa Y, Komiyama K, Tsuruta T, Murakami M, Masuda S, Akasaka Y, Ito K, Ishiguro S, Morita H, Sata S, Ishii T., “Expression of Secretary Phospholipase A2s in Human Atherosclerosis Development,” Atherosclerosis 2008 January; 196(1):81-91.
9) Wang W, Zhang K, Zhang H, Li M, Zhao Y, Wang B, Xin W, Yang W, Zhang J, Yue S, Yang X, “Underlying Genes Involved in Atherosclerotic Macrophages: Insights From Microarray Data Mining,” Med Sci Monit. 2019 Dec. 25; 25:9949-9962, doi: 10.12659/MSM.917068.
10) Tabibiazar R, Wagnere R A, Ashley E A, King J Y, Rerrara R, Spin J M, Sanan D A, Narasimhan B, Tibshirani R, Tsao P S, Efron B, Quertemous T, “Signature Patterns of Gene Expression in Mouse Atherosclerosis and Their Correlation to Human Coronary Disease,” Physiol Genomics. 2005; 22(2):213-226.
11) Chen W, Song T, Cao Q, Li R, Wang H, Chen X, Chen Z, “Atherosclerosis Prediction by Microarray-Based DNA Methylation Analysis,” Exp Ther Med. 2020 September; 20(3):2863-2869.
12) Zhang X, Sun R, Liu L, “Potentially Critical Roles of TNPO1, RAP1B, ZDHHC17, and PPM1B in the Progression of Coronary Atherosclerosis Through Microarray Data Analysis,” J Cell Biochem. 2019 March; 120(3):4301-4311.
13) Balashanmugam M V, Shivanandappa T B, Nagarethinam S, Vastrad B, Vastrad C. Analysis of differentially expressed genes in coronary artery disease by integrated microarray analysis. Biomolecules 10:35; doi:10.3390/biom10010035, 2020.
14) Zhao Z, Li X, Gao C, Jian D, Hao P, Rao L, Li M. Peripheral blood circular RNA hsa_circ_0124644 can be sued as a diagnostic biomarker of coronary artery disease. Scientific Rep 7:39918; doi:10.1038/srep39918, 2017.
15) Kaplan, D, D Smith. Enzymatic amplification staining for flow cytometric analysis of cell surface molecules. Cytometry 40:81-85, 2000.
16) Kaplan, D, H Meyerson, K Lewandowska. High resolution immunophenotypic analysis of chronic lymphocytic leukemia cells by enzymatic amplification staining (EAS). Amer J. Clin. Path. 116:429-36, 2001.
17) Kaplan, D, D Smith, H Meyerson, N Pecora, K Lewandowska. CD5 expression by B lymphocytes and its regulation upon Epstein-Barr Virus transformation. Proc. Natl. Acad. Sci. USA. 98:13850-13853, 2001.
18) Kaplan D. Flow cytometric analysis of cells from persons with HIV-1 disease by enzymatic amplification staining (EAS). Cellular Aspects of HIV Infection. Eds. A. Cossarizza, D. Kaplan. Wiley, New York, pp 351-369, 2001.
19) Kaplan, D, W Husel, H Meyerson Immunophenotypic analysis with enhanced sensitivity of detection by enzymatic amplification staining (EAS). Clinics Lab. Med. 21:763-778 2001.
20) Meyerson, H, D Kaplan. Applications of Enzymatic Amplification in Immunophenotyping, Frontiers in Biosciences. 7:33-43, 2002.
21) Kaplan, D, D Smith. Ultra-sensitive detection of cell surface molecules by enzymatic amplification staining and flow cytometry. Leucocyte typing VII. Oxford University Press. New York. Pp 402-403. 2002.
22) Kaplan, D, W Husel, K Lewandowska. CD34 expression on platelets. Platelets. 14:83-87, 2003.
23) Kaplan, D. Enzymatic amplification staining for cell surface antigens. Current protocols in cytometry. Wiley. New York. Pp 6.14.1-6.14.11, 2003.
24) Kaplan, D, H Meyerson, W Husel, K Lewandowska, G MacLennan D cyclins in lymphocytes. Cytometry 63A:1-9, 2005.
25) Meyerson, H, G MacLennan, W Husel, W Tse, H Lazarus, D Kaplan. D cyclins in CD5⁺ B cell lymphoproliferative disorders: cyclin D1 and cyclin D2 identify diagnostic groups and cyclin D1 correlates with ZAP-70 expression in chronic lymphocytic leukemia. Amer J Clin Path 125:1-10, 2006.
26) Shi, C, Sakuma M, Mooroka, T, Liscoe, A, Gao, H, Croce, K J, Sharma, A, Kaplan, D, Greaves, D R, Wang, Y, Simon, D I. Downregulation of the forkhead transcription factor Foxp1 is required for monocyte differentiation and macrophage function. Blood 112:4699-4711, 2008.
27) Kaplan, D, H I Meyerson, X Li, C Drasny, F Liu, M Costaldi, P Barr, H Lazarus. Correlation between ZAP-70, phospho-ZAP-70, and phospho-Syk expression in leukemic cells from patients with CLL. Cytometry B 78:115, 2010.
28) Lazarus H M, Sommers S R, Arfons L M, Fu P, Ataergin S A, Kaye N M, Liu F, Kindwall-Keller T L, Cooper B W, Laughlin M J, Creger R J, Barr P M, Gerson S L, 15. Kaplan D. Spontaneous autologous graft-versus-host disease in plasma cell myeloma autograft recipients: flow cytometric analysis of hematopoietic progenitor cell grafts. Biol Blood Marrow Transplant 17:970-978, 2011.
29) Kaplan D, Kaye N, Liu F, Fu P, Margevicius S, Meyerson H J, Lazarus H M. The functional duality of HoxB4 in hematopoietic reconstituting cells. Cytometry A 83A:127-133, 2013.
30) Kaplan, D, Sun Z, Tallman M S, Flinn 1W, Xiao W, Caimi P F, Kaye N M, Lazarus H M. Prognostic information and biological insights in chronic lymphocytic leukemia by high-resolution immunophenotypic analysis of ZAP70. Cytometry A 85A:798-808, 2014.
31) Mitchell, W B, Pinheiro M P, Boulad N, Kaplan D, Edison M N, Psaila B, Karpoff M, White M J, Josefsson E C, Kile B T, Bussel J B. Effect of thrombopoietin receptor agonists on the apoptotic profile of platelets in patients with chronic immune thrombocytopenia. Amer J Hematol. 89:E228-E234, 2014.

What is claimed is:

1. A method for detecting, in one or more peripheral blood cells from a subject having or at risk of having atherosclerosis, the level of gene expression of one or more components of the STING pathway in said cells, the method comprising detecting the level of gene expression of said components in said cells.

2. The method of claim 1, wherein the peripheral blood cells are mononuclear cells.

3. The method of claim 1, wherein the peripheral blood mononuclear cells comprise one or more of B lymphocytes, T lymphocytes, monocytes and natural killer (NK) cells.

4. The method of claim 3, wherein the T lymphocytes are CD8⁺ T lymphocytes or CD4⁺ T lymphocytes.

5. The method of claim 1, wherein the component is one or more of STING, phospho-STING, Akt, phospho-Akt, IRF3, phospho-IRF3, TBK1, phospho-TBK1, ULK1, phospho-ULK1, cGAS, NLRP3, RelA, phospho-RelA, BDNF, VEGF, MyD88, Trex1, AIM2, Caspase1, SURF4, STEEP1, STIM1, HER2, Stat6 and Src.

6. The method of claim 1, wherein detection comprises one or more of expression of:
STING in monocytes; phospho-STING in monocytes, phospho-STING in B lymphocytes, phospho-STING in CD4⁺ T lymphocytes, phospho-STING in CD8⁺ T lymphocytes; IRF3 in monocytes, IRF3 in CD4⁺ T lymphocytes, IRF3 in CD8⁺ T lymphocytes; BDNF in monocytes, BDNF in B lymphocytes, BDNF in CD4⁺ T lymphocytes, BDNF in CD8⁺ T lymphocytes; phospho-RelA in CD4⁺ T lymphocytes, phospho-RelA in CD8⁺ T lymphocytes; phospho-TBK1 in monocytes, phospho-TBK1 in B lymphocytes, phospho-TBK1 in CD4⁺ T lymphocytes, phospho-TBK1 in CD8⁺ T lymphocytes; phospho-Akt in monocytes, phospho-Akt in B lymphocytes; MyD88 in monocytes; and NLRP3 in monocytes, NLRP3 in B lymphocytes, NLRP3 in CD4⁺ T lymphocytes, and NLRP3 in CD8⁺ T lymphocytes.

7-34. (canceled)

35. The method of claim 1, wherein the detection assay comprises detecting a co-stain on individual cells wherein the co-stain is for (I) a cell lineage marker for a peripheral blood cell and (II) one or more components of the STING pathway.

36. The method of claim 35, wherein the cell lineage marker is a marker selected from the group consisting of a marker of B lymphocytes, T lymphocytes, monocytes and natural killer (NK) cells.

37. The method of claim 35, wherein the component is selected from the group consisting of STING, phospho-STING, Akt, phospho-Akt, IRF3, phospho-IRF3, TBK1, phospho-TBK1, ULK1, phospho-ULK1, cGAS, NLRP3, RelA, phospho-RelA, BDNF, VEGF, MyD88, Trex1, AIM2, Caspase1, SURF4, STEEP1, STIM1, HER2, Stat6 and Src.

38-40. (canceled)

41. A method for testing a compound for its effects on atherosclerosis, the method comprising administering a compound to a subject having or at risk for having atherosclerosis and detecting the effect of the compound on the development, progression or severity of the disease, the compound being an inducer or inhibitor of gene expression of one or more components of the STING pathway.

42. (canceled)

43. The method of claim 41, wherein the component is selected from the group consisting of STING, phospho-STING, Akt, phospho-Akt, IRF3, phospho-IRF3, TBK1, phospho-TBK1, ULK1, phospho-ULK1, cGAS, NLRP3, RelA, phospho-RelA, BDNF, VEGF, MyD88, Trex1, AIM2, Caspase1, SURF4, STEEP1, STIM1, HER2, Stat6 and Src.

44. A method to treat a subject having atherosclerosis comprising administering an inducer or inhibitor of one or more components of the STING pathway.

45. (canceled)

46. The method of claim 44, wherein the component is selected from the group consisting of STING, phospho-STING, Akt, phospho-Akt, IRF3, phospho-IRF3, TBK1, phospho-TBK1, ULK1, phospho-ULK1, cGAS, NLRP3, RelA, phospho-RelA, BDNF, VEGF, MyD88, Trex1, AIM2, Caspase1, SURF4, STEEP1, STIM1, HER2, Stat6 and Src.

47. A composition comprising peripheral blood cells in which individual cells are co-stained for a cell-lineage marker for the peripheral blood cell and for a marker for one or more components of the STING pathway.

48. The composition of claim 47 comprising more than one peripheral blood cell type.

49. The composition of 47 wherein the peripheral blood cell is a mononuclear cell.

50. The composition of 49 wherein the mononuclear cell is selected from the group consisting of monocytes, B cells, and T cells.

51-59. (canceled)

60. The composition of claim 47 wherein the marker for a component of the STING pathway is selected from the group consisting of STING, phospho-STING, Akt, phospho-Akt, IRF3, phospho-IRF3, TBK1, phospho-TBK1, ULK1, phospho-ULK1, cGAS, NLRP3, RelA, phospho-RelA, BDNF, VEGF, MyD88, Trex1, AIM2, Caspase1, SURF4, STEEP1, STIM1, HER2, Stat6 and Src.

61. (canceled)

62. A kit containing an antibody to one or more cell-lineage markers on peripheral blood cells, an antibody to one or more STING pathway components, a cell-fixation reagent and a cell-permeabilization reagent.

63-64. (canceled)

65. The kit of claim 62, wherein the one or more STING pathway components include STING, phospho-STING, Akt, phospho-Akt, IRF3, phospho-IRF3, TBK1, phospho-TBK1, ULK1, phospho-ULK1, cGAS, NLRP3, RelA, phospho-RelA, BDNF, VEGF, MyD88, Trex1, AIM2, Caspase1, SURF4, STEEP1, STIM1, HER2, Stat6 and Src.
